(19) United States
(12) Patent Application Publication
(10) Pub. No.: US 2024/0271213 A1
(43) Pub. Date: Aug. 15, 2024

(54) METHODS AND COMPOSITIONS COMPRISING FUNCTIONAL GENOMICS OF HIBERNATING MAMMALS

(71) Applicant: Fauna Bio Incorporated, Emeryville, CA (US)

(72) Inventors: Katharine R. Grabek, Benicia, CA (US); Linda Goodman, San Francisco, CA (US); Ashley Zehnder, Emeryville, CA (US); Gokmen Altay, Emeryville, CA (US)

(21) Appl. No.: 18/568,603

(22) PCT Filed: Jun. 9, 2022
(86) PCT No.: PCT/US22/32854

§ 371 Date: Dec. 8, 2023

Related U.S. Application Data
(60) Provisional application No. 63/208,898, filed on Jun. 9, 2021.

(51) Int. Cl. C12Q 1/6883; G01N 33/50

(52) U.S. Cl. CPC C12Q 1/6883; G01N 33/5008; C12Q 2600/136; C12Q 2600/158

(57) ABSTRACT

Methods of drug target and therapeutic compound discovery utilizing human disease-related phenotypes in hibernating mammals followed by target validation. Drug target identification of genes is accomplished by identification of adaptations in hibernating mammals using molecular interaction databases. and confirmation of disease relevance of identified genes across other animals followed by in vitro validation. Methods of identifying a therapeutic target by obtaining tissue derived from one or more hibernator mammal species: generating a gene expression profile from each tissue: matching the gene expression profiles of hibernator animal to generate a common gene expression signature: identifying a candidate gene target from the common gene expression signature: wherein the candidate gene target is found to be differentially expressed in tissues from a hibernating animal obtained when the animal is protected from a disease or condition compared to tissues obtained from a hibernating animal at a time when the hibernating animal is not protected.

### CROSS REFERENCE TO RELATED APPLICATIONS

This application claims the benefit of U.S. Provisional Application No. 63/208,898, filed Jun. 9, 2021, which is hereby incorporated in its entirety by reference for all purposes.

### SEQUENCE LISTING

Not applicable.

### BACKGROUND

The genomics era has brought about a fundamental shift in the way new drugs are designed, allowing researchers to study humans as biological systems that can be adjusted to cure a disease based on pre-defined genetic targets. However, curing complex diseases is an intricate process and many low-hanging genomic fruits in humans have already been targeted by multiple drugs. Drug design efforts often focus on a very small percentage of the human genome that has already been intensively studied, while many animals with human disease-related phenotypes remain understudied.

A highly effective approach to target identification is to study naturally occurring genetic resistance to a disease. Yet humans are a limited resource for identifying disease-resistance mutations, and much greater diversity exists if we broaden our search throughout animals. To fully reap the benefits of the genomics era, research will need to look beyond the well-known pathways identified in humans and a limited number of model organisms. Particularly amenable to target identification are species at temporary high risk for problems that closely mirror human diseases but who are able to completely avoid or even reverse transient pathologies.

Hibernating mammals are a treasure trove of transient genetic resistance information whose state can be monitored with body temperature. They are able to completely prevent or reverse pathologies that closely resemble human diseases, such as: Alzheimer's disease. ischemia-reperfusion damage from heart attack and stroke. obesity/diabetes, muscle loss and bone loss. For Alzheimer's disease, upon entering torpor during hibernation, cell bodies, spines, and dendrites from diverse neuronal cell types in hibernating ground squirrels retract and hyperphosphorylated tau tangles accumulate, as during Alzheimer's disease (Dave et al., 2003). Yet, when animals arouse from torpor, neurons re-grow and the tau proteins are cleared (Arendt et al., 2003). For ischemia-reperfusion damage from heart attack and stroke, while in deep torpor, model hibernators such as ground squirrels have a heart rate at 1% of normal, and their body temperature drops to 4° C. At least 25 times during winter hibernation, they rapidly rewarm to 37° C. in just 2-3 hours; remarkably, heart rate increases 100-fold by the time core body temperature reaches just 7° C. (MacCannell et al., 2018). This phenotype mimics an ischemia-reperfusion event similar to a heart attack or stroke, yet they are protected from the associated tissue damage. Studies show that when surgically simulating an ischemia-reperfusion event, winter squirrels are largely protected from damage compared to summer squirrels (reduced plasma levels of troponin I, myocardial apoptosis, and left ventricular contractile dysfunction) (Quinones et al., 2016). For obesity/diabetes, hibernators are hyperphagic during the summer, becoming obese and insulin resistant in the fall prior to hibernation. They then cease all food intake for the entire 6-9 months of winter hibernation, emerging in the spring thin and insulin sensitive (Martin et al, 2008). For muscle loss and bone loss, despite lying nearly motionless for 6-9 months of the year, hibernators emerge ready to run from predators, displaying a remarkable ability to maintain muscle and bone mass despite extended periods of disuse (Andres-Mateos et al., 2012);(Utz et al., 2009). However, the gene expression changes that produce all of the above transient effects on disease have yet to be interrogated for drug target discovery.

Thus, to fully reap the benefits of the genomics era, an understanding and elucidation of the mechanisms in hibernating animals and the underlying resistance to diseases afflicting humans, ranging from neurodegenerative, cardiovascular and metabolic diseases is needed.

### SUMMARY

In certain aspects, disclosed herein are methods of identifying a therapeutic target that can be modulated to treat a disease or condition, comprising obtaining tissue derived from one or more of an hibernator mammal species; generating a gene expression profile from each tissue; matching the gene expression profile from each tissue of hibernator animal to generate a common gene expression signature; identifying a candidate gene target from the common gene expression signature; wherein the candidate gene target is found to be differentially expressed in tissues from a hibernating animal obtained when the animal is protected from a disease or condition compared to tissues obtained from a hibernating animal at a time when the hibernating animal is not protected from the disease or condition. In certain embodiments, the tissue is harvested at a time during the mammal's hibernating cycle when the hibernating animal is protected from the disease or condition. In certain embodiments, a plurality of tissue samples are each harvested at discrete body temperature intervals during the torpor-arousal cycle and time intervals before, during or after the mammal's hibernating cycle. In certain embodiments, the methods further comprise performing an assay, wherein the assay comprises contacting cells with a modulator of the candidate gene target and determining if a desired cellular response is generated. In certain embodiments, the methods further comprise identifying a gene as a therapeutic target for the disease or condition if the desired cellular response is observed after contacting the cells with the inhibitor; wherein the desired cellular response indicates reduced cellular indicators of the disease.

In certain aspects, described herein are methods of identifying a therapeutic compound for treating a disease or condition, comprising obtaining a gene expression profile from one or more of a hibernator mammal species, wherein the gene expression profile reflects discrete body temperature intervals during the torpor-arousal cycle and time intervals before, during or after the mammal's hibernating cycle; identifying a candidate gene target from the gene expression profile; and contacting mammalian cells having the candidate gene target with a candidate compound, and measuring the gene activity of the cell.

In certain aspects, described herein are methods for identifying a therapeutic compound for the treatment of a disease or condition, comprising obtaining tissue derived from each of a plurality of hibernating animals; wherein the tissue is harvested at a time point during the animal's hibernating cycle when the hibernating animal is protected from the disease or condition; generating a gene expression profile from each tissue; matching the gene expression profile from each tissue of the plurality of hibernating animals to generate a common gene expression signature; identifying at least one gene target from the common gene expression signature; wherein the gene target is found to be differentially expressed in the tissues from a hibernating animal obtained when the animal is protected from a disease or condition compared to tissues obtained from a hibernating animal at a time-point when the hibernating animal is not protected from the disease or condition; contacting cells having the gene target with a candidate therapeutic; and determining if a desired cellular response is generated after contacting the cells with the candidate therapeutic compound.

In certain embodiments of the methods described herein, the disease or condition is selected from the group consisting of ischemia-reperfusion injury, neurovascular diseases, stroke, neurodegeneration, Alzheimer's disease, traumatic brain injury, thromboembolic disease, peripheral vascular disease, cardiovascular disease, obesity, and diabetes, muscle atrophy, sarcopenia, muscle wasting, bone atrophy, osteoporosis, osteopenia, inflammation, suspended animation, seasonal affective disorder, hyperthyroidism, hypothyroidism, diseases stemming from increased intestinal permeability, chronic fatigue syndrome, chronic obstructive pulmonary disease (COPD), fibrotic diseases, idiopathic pulmonary fibrosis (IPF), non-alcoholic steatohepatitis (NASH), non-alcoholic fatty liver disease (NAFLD), and ionizing radiation. In certain embodiments, the disease or condition is cardiac disease. In certain embodiments, the disease or condition is neuronal ischemia/reperfusion injury. In certain embodiments, the candidate gene target is selected from a gene in Table 1 or Table 2.

In certain embodiments, of the methods described herein, the generating the gene expression signature comprises performing RNA SEQ. In certain embodiments, the generation of the gene expression signature further comprises performing a method selected from the group consisting of: epigenomic analysis, ATAC-seq, proteomic analysis, quantitative PCR and DNA sequencing and combinations thereof.

In certain embodiments, the candidate gene target is undergoing accelerated evolution in at least one hibernating mammal as compared to non-hibernating mammals. In certain embodiments, the candidate gene target is conserved in hibernating mammals as compared to non-hibernating mammals.

In certain embodiments, the methods further comprise analysis of human genome-wide association study (GWAS) data to identify the disease or condition that is associated with a candidate gene target.

In certain embodiments of the methods described herein, the hibernating mammal is selected from the group consisting of: (Ictidomys tridecemlineatus, Urocitellus parryii, Marmota monax, Spermophilus dauricus, Tenrec ecaudatus, Cheirogaleus medius, Cheirogaleus crossleyi, Cheirogaleus sibreei, Dromiciops gliroides, Cercartetus nanus, Burramys parvus, Tachyglossus aculeatus, Mirza coquereli, Glis glis, Graphiurus murinus, Muscardinus avellanarius, Miniopterus schreibersii, Rhinolophus ferrumequinum, Zapus hudsonius, Mesocricetus auratus, Cricetus cricetus, Erinaceus europaeus, Ursus arctos, Ursus americanus, Miniopterus natalensis, Myotis brandtii, Eptesicus fuscus, Myotis myotis, Myotis brandtii, Myotis ricketti and Myotis lucifugus).

In certain embodiments, the obtaining of tissues from each of a plurality of hibernating mammals is from different species. In certain embodiments, the desired cellular response is selected from the group consisting of: reduced cell death, increased cell survival, decreased cell damage, reduced oxidative stress and decreased inflammation. In certain embodiments, the cells are human cells. In certain embodiments, the modulator of the candidate gene target is one of a small molecule, a nucleic acid or a protein. In certain embodiments, the nucleic acid is RNA. In certain embodiments, the RNA is antisense RNA.

In certain embodiments, the candidate therapeutic compound changes the expression of at least one gene target. In certain embodiments, the candidate gene target is selected from a gene in Table 1 or Table 2. In certain embodiments, the candidate therapeutic compound causes a cellular response of increased cell survival, reduced oxidative stress, reduced reactive oxygen species, decreased cell damage, decreased inflammation or combinations thereof, after the cells have been subjected to a condition selected from the group consisting of: hypoxia, starvation, low glucose, oxidative stress, and reduced temperature. In certain embodiments, the increased survival cell survival is caused by reduced apoptosis.

In certain embodiments, the methods further comprise identifying a plurality of target genes that functionally interact in a common cellular pathway; wherein the candidate therapeutic compound changes the expression of at least one of the target genes of the cellular pathway or changes the activity of a protein expressed by at least one of the target genes in the cellular pathway.

In certain embodiments, the therapeutic compound is administered to a patient in a sufficient amount for treatment of neuronal injury following a stroke. In certain embodiments, the therapeutic compound is administered to a patient in a sufficient amount for treatment of a cardiovascular disease. In certain embodiments, the therapeutic compound is administered to a patient in a sufficient amount for treatment of Alzheimer's disease. In certain embodiments, the candidate therapeutic compound causes increased cell viability, reduced reactive oxygen species, reduced cell damage, reduced inflammation or combinations thereof in neurons, after the neurons have been subjected to hypoxic conditions. In certain embodiments, the increased cell viability is caused by decreased apoptosis. In certain embodiments, the candidate therapeutic compound causes increased cell survival in neurons subjected to Aβ1-42 toxicity.

In certain aspects, described herein is method of identifying a therapeutic target that can be modulated to treat a disease or condition, comprising: computing an association between an input gene expression signature and compounds in a previously determined dataset to nominate candidate therapeutics that can induce or reverse the signature, wherein the input gene expression signature represents changes in gene expression in a cell line; determining the similarity between the input gene expression signature and each signature in a database using similarity metrics; normalizing the similarity scores by comparing them to reference similarity distributions; and identifying a candidate gene target based on the normalized similarity score. In certain embodiments, the previously determined dataset comprises a database of gene expression profiles that characterizes a plurality of compounds by the gene expression changes the compounds induce in cell lines. In certain embodiments, the similarity metric comprises a weighted connectivity score or cosine distance. In certain embodiments, the generating comprises generating a single answer for each compound-cell line pair tested. In certain embodiments, the methods further comprise summarizing normalized scores within and across cell lines using a set of order statistics compared to a reference distribution.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 is a graph illustrating the hibernation cycle for the model hibernator, the 13-lined ground squirrel.

FIG. 2A is a graph of the average body temperature of the 13-lined ground squirrel.

FIG. 2B are images of magnified thalamic neurons illustrating that during Entrance, neuronal spines retract (white arrows) and hyperphosphorylated tau angles develop, and during Arousal, neuronal connections regrow and tau proteins are cleared.

FIG. 2C is a graph illustrating ischemia-reperfusion phenotype of echocardiographic left ventricular systolic function (LV-FAC) after surgically-induced ischemia reperfusion events in winter hibernating squirrels compared to summer non-hibernating squirrels.

FIG. 3 is a heat map illustrating gene expression profiles generated from three tissues: heart, hypothalamus and intestines of 34 squirrels at different timepoints during the hibernation cycle using RNA-SEQ.

FIG. 4A is a graph illustrating gene expression of genes known to be correlated with cardiac protection. RNA-SEQ was performed from heart tissues of squirrels at different timepoints of the hibernation cycle. Expression of six genes: IBA, Ent, ET, LT, EAr, Lar was obtained by RNA-SEQ from Squirrels in Winter (hibernating animals), Spring (transitional phase) and Summer (non-hibernating animals).

FIG. 4B is a graph illustrating gene expression of genes known to be correlated with cardiac protection. RNA-SEQ was performed from heart tissues of squirrels at different timepoints of the hibernation cycle. Expression of six genes: IBA, Ent, ET, LT, EAr, Lar was obtained by RNA-SEQ from Squirrels in Winter (hibernating animals), Spring (transitional phase) and Summer (non-hibernating animals).

FIG. 5 is a graph illustrating accelerated evolution of groups of genes associated with various physiological mechanisms or pathologies in hibernators compared to non-hibernating animals.

FIG. 6 is a flow-chart illustrating the target identification and drug discovery platform comprising multi-omics data sets, computational modeling and in vitro target validation.

FIG. 7A is an illustration of a gene network module assembled for downregulated genes to identify additional correlated genes. Labeled genes Target Numbers (TN) 15 and 34 are two correlated candidate gene targets identified for in vitro validation.

FIG. 7B is an illustration of a gene network module assembled for downregulated genes to identify additional correlated genes. Labeled genes Target Numbers (TN) 56 and 26 are two correlated candidate gene targets identified for in vitro validation.

FIG. 8 is a flowchart illustrating the process for scoring potential candidate gene targets.

FIG. 9A is a heatmap and table showing select top scoring candidate therapeutic compounds obtained for cardioprotection from ischemia reperfusion injury using genetic perturbation data, including compounds known to have protective activity against cardiac pathologies.

FIG. 9B is a diagram showing results of comparison of select top scoring candidate therapeutic compounds for cardioprotection using genetic perturbation data (compounds that can mimic gene expression signatures of hibernators identified as cardioprotective), accelerated evolution (evolutionary rate) analysis and human GWAS data analysis.

FIG. 10 is a heatmap diagram showing differential gene expression and multi-omics integrated data for identifying candidate therapeutic compounds for Alzheimer's Disease protection by identifying compounds that can mimic gene expression signatures of hibernators identified as neuroprotective.

FIG. 11 is a table showing select top scoring gene targets obtained for Alzheimer's Disease protection, including compounds known to have neuroprotective activity.

FIG. 12 is a diagram illustrating a candidate gene target identified for cardiac protection by analysis of RNA-seq and ATAC-seq data across tissues. ATAC-seq analysis was performed per peak (have different levels of chromatin structure within a gene) and within 10Kb of the TN 15 gene.

FIG. 13A is a graph showing cell viability as detected by MTT assay of AC16 cells with FAUN103G (Target Number 15) gene knockdown after 16 hrs ischemia and 4 hrs reperfusion.

FIG. 13B is a graph showing LDH activity in AC16 cells with Target Number (TN) 15 gene knockdown after 16 hrs. ischemia and 4 hrs reperfusion.

FIG. 13C is a graph showing cell viability as detected by MTT assay of AC16 cells with Target Number (TN) 34 gene knockdown after 16 h ischemia and 4 h reperfusion.

FIG. 13D is a graph showing LDH activity in AC16 cells with Target Number (TN) 34 gene knockdown after 16 h ischemia and 4 h reperfusion.

FIG. 14A is a graph showing cell viability as detected by MTT assay of AC16 cells with Target number (TN) 56 gene knockdown after 16 h ischemia and 4 h reperfusion.

FIG. 14B is a graph showing LDH activity in AC16 cells with Target number (TN) 56 gene knockdown after 16 h ischemia and 4 h reperfusion.

FIG. 14C is a graph showing cell viability as detected by MTT assay of AC16 cells with Target number (TN) 26 gene knockdown after 16 h ischemia and 4 h reperfusion.

FIG. 14D is a graph showing LDH activity in AC16 cells with Target number (TN) 26 gene knockdown after 16 h ischemia and 4 h reperfusion.

FIG. 15A is a graph showing cell viability as detected by MTT assay of AC16 cells with Target number (TN) 38 gene knockdown after 16 h ischemia and 4 h reperfusion.

FIG. 15B is a graph showing LDH activity in AC16 cells with Target number (TN) 38 gene knockdown after 16 h ischemia and 4 h reperfusion.

FIG. 15C is a graph showing cell viability as detected by MTT assay of AC16 cells with Target number (TN) 49 gene knockdown after 16 h ischemia and 4 h reperfusion.

FIG. 15D is a graph showing LDH activity in AC16 cells with Target number (TN) 49 gene knockdown after 16 h ischemia and 4 h reperfusion

FIG. 16 are images of human SH-SY5Y neuroblastoma cells (left) differentiated into mature neurons (right) by incubating cells in retinoic acid and N₂ supplement for 9 days. Arrow indicates long neurite typical of differentiated neurons.

FIG. 17 is a graph illustrating results of an in vitro validation assay for ischemia-reperfusion injury in H9C2 cells. H9C2 cells were exposed to ischemia for 17 hrs and reperfusion for 4 hrs were contacted with differing concentrations of melatonin at the start of ischemia. Viability was measured using the MTT assay. Data are expressed as the mean +/− standard deviation in experiments performed in triplicate. * p<0.05 and ** p<001 compared to vehicle control.

FIG. 18A is a graph showing in vitro cell viability of AC16 cells following 16 hrs ischemia and 4 hrs reperfusion and incubated with different concentrations of a compound identified for neuroprotection, Triptolide.

FIG. 18B is a graph showing a decrease in cell damage response (LDH activity) of AC16 cells following 16 hrs ischemia and 4 hrs reperfusion and incubated with different concentrations of Triptolide.

FIG. 18C is a graph showing a decrease production of reactive oxygen species (ROS) of AC16 cells following incubation with Triptolide.

FIG. 19 is a graph showing an increase in cell viability following incubation with two compounds predicted from gene modules, and an approved Alzheimer's drug, Memantine.

FIG. 20 is a graph showing signatures of the same compound as a query signature tend to be most similar with LEO. The x-axis is the rank of the matched compound signatures to 25 query signatures, and the y-axis is the cumulative fraction. All four methods in LEO tend to find matched-compound signatures in a query against the database despite cell line, time point, and dose confounders. The numbers in the lower right are the auROCs of the four LEO methodologies. The grey dotted line on y=x indicates the performance of an uninformative classifier.

FIG. 21 is a graph showing the summary of auROCs for matched compound signatures (left) and summary scores for compounds in the same pharmacological class (PCL, right) or with the same mechanism of action. The values shown are the area under the receiver operating characteristic curve. An auROC of 0.5 is equivalent to a random classifier.

FIG. 22 is a graph showing recall rates for benchmark queries for matched compound signatures and summary scores for compounds with the same mechanism of action. This analysis looks at the top 5% of signatures similarities (left) and summarized compound scores (right) and counts the fraction of expected positives in that top 5%. All the LEO metrics show better than expected recall; an uninformative classifier would have recall of 5%.

FIG. 23 is a diagram depicting the upregulated genes from Module 16.

FIG. 24A is a diagram depicting the experimental design for echochardiography from an in vivo LAD ligation study/

FIG. 24B is a graph depicting left ventricle (LV) ejection fraction resulting from the in vivo LAD ligation study evaluating intracardiac injection of Faun 1003.

FIG. 24C is a graph depicting fractional shortening resulting from the in vivo LAD ligation study evaluating intracardiac injection of Faun1003.

FIG. 24D is a graph depicting cardiac output resulting from the in vivo LAD ligation study evaluating intracardiac injection of Faun 1003.

### DETAILED DESCRIPTION

### Definitions

Terms used in the claims and specification are defined as set forth below unless otherwise specified.

The term “ameliorating” refers to any therapeutically beneficial result in the treatment of a disease or disorder state, e.g., a cardiovascular disease state, including prophylaxis, lessening the severity or progression, remission, or cure thereof.

The term “in-vitro ” refers to processes that occur in a living cell growing separate from a living organism, e.g., growing in tissue culture.

The term “in vivo” refers to processes that occur in a living organism.

The term “mammal” as used herein includes both humans and non-humans and include, but is not limited to humans, non-human primates, canines, felines, murines, bovines, equines, and porcines.

The term “hibernating animal,” “hibernating mammal,” or “hibernator” refers to any hibernating mammal including, but not limited to, (Ictidomys tridecemlineatus, Urocitellus parryii, Marmota monax, Spermophilus dauricus, Tenrec ecaudatus, Cheirogaleus medius, Cheirogaleus crossleyi, Cheirogaleus sibreei, Dromiciops gliroides, Cercartetus nanus, Burramys parvus, Tachyglossus aculeatus, Mirza coquereli, Glis glis, Graphiurus murinus, Muscardinus avellanarius, Miniopterus schreibersii, Rhinolophus ferrumequinum, Zapus hudsonius, Mesocricetus auratus, Cricetus cricetus, Erinaceus europaeus, Ursus arctos, Ursus americanus, Miniopterus natalensis, Myotis brandtii, Eptesicus fuscus, Myotis myotis, Myotis brandtii, Myotis ricketti and Myotis lucifugus).

The term “hibernating cycle” refers to characteristic seasonal phases of the hibernator's year, which oscillate between a period of extended heterothermy or hibernation and a period of extended homeothermy; these can be identified by the time of year and magnitude of change in the animal's body temperature.

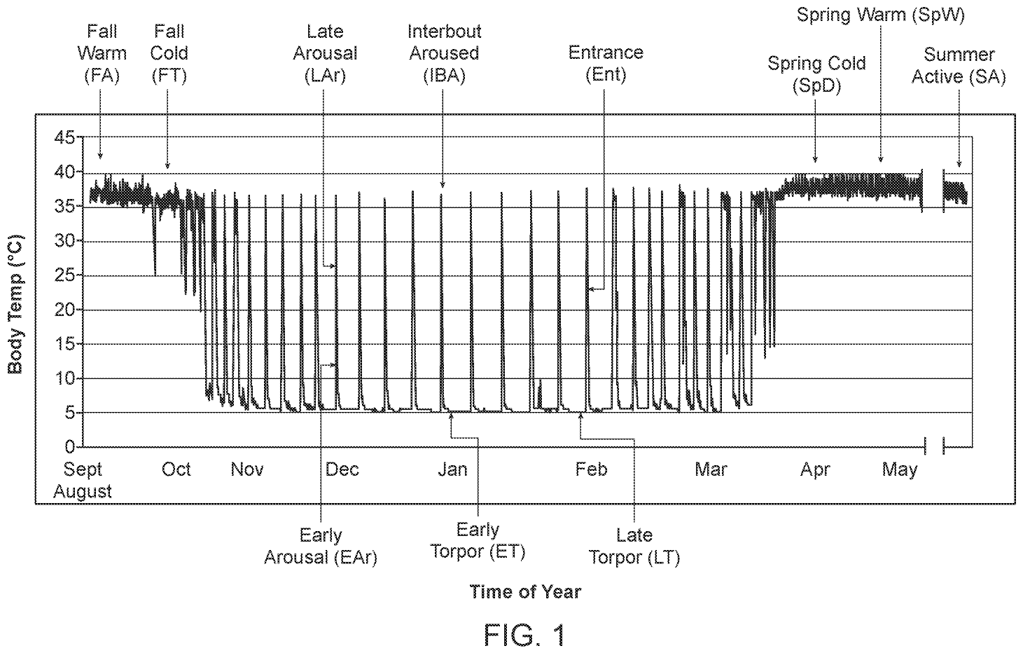

The term “torpor-arousal cycle” refers to the characteristic phases within the heterothermic or hibernating portion of the year of a deep hibernating animal, which can be identified by the hibernating animal's body temperature. For example, FIG. 1 shows an example hibernation cycle for the 13-lined ground squirrel.

The term “sufficient amount” means an amount sufficient to produce a desired effect, e.g., an amount sufficient to modulate the symptoms of or onset of cardiovascular disease in a human patient.

The term “therapeutically effective amount” is an amount that is effective to ameliorate a symptom of a disease.

The term “cardioprotection”, “cardiovascular protection”, or “cardiac protection” refers to gene targets or compounds that improve symptoms and/or reduce indicators associated with a cardiovascular injury, disease and/or condition including, but not limited to, ischemia-reperfusion injury, thromboembolic disease, peripheral vascular disease, cardiovascular disease, obesity, and diabetes.

The term “neurological protection”, “neuronal injury protection”, or “neuroprotection” refers to gene targets or compounds that improve symptoms and/or reduce indicators associated with a neuronal and/or nervous system injury, disease or condition including, but not limited to, stroke, neurodegeneration, Alzheimer's disease, and traumatic brain injury.

The term “desired cell response” refers to any cellular response or behavior that are related to a disease or disorder, including, but not limited to, cellular behaviors that indicate a reduction or reversal in cellular physiology, and/or cellular signaling that is associated with a disease or disorder.

The term “gene expression profile” refers to any data that relates to the expression or activity of a plurality of genes.

The term “common gene expression signature” refers to a gene expression signature (e.g., a list of a plurality of genes that are over or under expressed) obtained by integrating gene expression profiles from each of a plurality of tissue samples, either from a single species or from a plurality of species, so that the common gene expression signature represents the genes that are significantly differentially expressed across the plurality of tissue samples.

The term, “genetic conservation,” refers to genes that share similar sequence across species.

The term, “differentially expressed” refers to genes whose expression in a tissue sample is altered (either increased or decreased) compared to genes of a reference sample. A gene can be differentially expressed if there is any increase or decrease in expression (e.g., amount of mRNA or cDNA) including any difference of 1.2-fold, 1.5-fold, 2-fold, 3-fold, 4-fold, 5-fold, 10-fold, 20-fold, 50-fold, 100-fold, 200-fold, 500-fold, 1000-fold, or more.

The term, “multi-omics” refers to: a plurality of functional genomics assays or data (e.g., data sets) obtained from such plurality of functional genomics assays. Functional genomics assays include, but are not limited to, transcriptomic, epigenomic, proteomic, and metabolomic assays such as, but not limited to, RNA-seq ATAC-seq, microarray, Genome-Wide Association Studies (GWAS), differential gene expression (DGE) analysis, mass spectrometry, immunoassays, and protein microarrays.

The term, “accelerated evolution” refers to genes that are conserved throughout mammals but are evolving rapidly in specific lineages. Some genes of particular importance to hibernation will have accelerated substitution rates (above neutrality) across multiple clades of hibernators or torpor-capable mammals. In certain embodiments, UCSC's neutral phylogenetic model for mammals is used and genes with accelerated substitution rates identified with the phyloP software [REF-phyloP].

It must be noted that, as used in the specification and the appended claims, the singular forms “a,” “an” and “the” include plural referents unless the context clearly dictates otherwise.

This application is generally directed in part to methods that include the use of hibernating animal models to address two key difficulties with genomically-driven drug design: target novelty and translatability to humans. Thus, described herein are methods of drug target discovery that utilize human disease-related phenotypes in hibernating mammals followed by target validation across multiple species and data types.

### Differential Gene Expression and Multi-Omics Approaches

The methods described herein utilize samples from a model hibernator (e.g., the 13-lined ground squirrel), to observe the gene expression signatures that occur when animals are being transiently protected from a disease or condition or when pathologies are being reversed, which does not occur in humans.

In certain aspects, described herein are methods of drug target identification of novel genes identified through adaptations in hibernating mammals, comprising generation of gene networks obtained from differential gene expression analysis, improvement of gene networks using molecular interaction databases, and confirmation of disease relevance of identified genes across other animals and data types followed by in vitro validation. In certain embodiments, the methods described herein also increase the likelihood that the identified targets will translate into successful human therapies.

In certain aspects, the application describes methods of identifying a therapeutic target that can be modulated to treat a disease or condition, comprising: obtaining tissue derived from one or more of a hibernator mammal species; generating a gene expression profile from each tissue; matching the gene expression profile from each tissue of hibernator animal to generate a common gene expression signature from animals sacrificed at disease-relevant hibernation time points; gene expression profiles are determined with analysis in DESeq2 or similar software; identifying a candidate gene target from the common gene expression signature; wherein the candidate gene target is found to be differentially expressed compared to tissues obtained from a hibernating animal at a time when the hibernating animal is not protected from the characteristic that is similar to a human disease or condition.

In certain aspects, the application describes methods of identifying a therapeutic compound for treating a disease or condition, comprising: obtaining a gene expression profile from one or more of a hibernator mammal species, wherein the gene expression profile reflects discrete body temperature intervals during the torpor-arousal cycle and time intervals before, during or after the mammal's hibernating cycle; identifying a candidate gene target from the gene expression profile; contacting mammalian cells having the candidate gene target with a candidate compound, and measuring the gene activity of the cell.

In certain aspects, the application describes methods identifying a therapeutic compound for the treatment of a disease or condition, comprising: obtaining tissue derived from each of a plurality of hibernating animals; wherein the tissue is harvested at a time point during the animal's hibernating cycle when the hibernating animal is protected from the disease or condition; generating a gene expression profile from each tissue; matching the gene expression profile from each tissue of the plurality of hibernating animals to generate a common gene expression signature; identifying at least one gene target from the common gene expression signature; wherein the gene target is found to be differentially expressed compared to tissues obtained from a hibernating animal at a time-point when the hibernating animal is not protected from the disease or condition; contacting cells having the gene target with a candidate therapeutic; and determining if a desired cellular response is generated after contacting the cells with the candidate therapeutic compound.

In certain aspects, a common gene expression profile is generated by performing differential gene expression analysis among the hibernation states of a hibernating mammal within a single tissue, wherein detected hibernation specific genetic targets are the targets obtained from a specific tissue; repeating the differential analysis for each specific tissue separately, resulting in detection of hibernation specific genes for each tissue separately. Therefore, targets are detected that are differential with respect to the selected time points within samples of one tissue, thus avoiding differences between tissues.

In certain embodiments, the methods described herein comprise performing Principal Component Analysis (PCA) and hierarchical clustering to test if batch effects exist among the samples from the same tissue. For example, if batch effects are observed, the data are Log2 normalized and the ComBat function of the “sva” R programming package is used; the removeBatchEffect function of the “limma” R programming package could alternatively be used; the batch corrected datasets are used as input in the downstream analyses such as WGCNA and C3Net. For differential gene expression analysis, any observed batch effects are input as covariates into the likelihood ratio test in DESeq2. Since a separate analysis for each tissue is performed and their results are independent, batch effects between the tissues may not be considered.

In certain embodiments, classification and regression tools are used to classify samples by a hibernation state regardless of tissue type. In certain embodiments, the methods comprise performing Random Forests classification and regression tool to classify samples by hibernation state regardless of tissue as well as identify the set of features from the multi-omics dataset that best discriminate the hibernation states (the top classifiers). To account for tissue-specific effects, each feature of the input data is first normalized and mean-scaled across samples within each tissue. The scaled data is combined across all samples, where the hibernation state label for each sample is retained while tissue type information is discarded. Random Forests is then performed to classify samples into hibernation state regardless of tissue type. Because there are thousands of input features from the multi-omics data, a round of variable selection using the varSelRF package in R is performed to identify the minimum number of features needed to produce the least amount of error when classifying samples. Finally, Random Forests is performed with the Random Forest package in R using the subset of features identified from the initial round of variable selection. The top features from the regression in Random Forests is examined and input into the combined analysis to create a short list of potential drug targets.

In certain embodiments, if the integrated multi-tissue approach does not adequately correct for tissue differences, as determined by inspection of sample clustering, Random Forests is run separately for each tissue using only multi-omics data from that tissue. The resulting top Random Forests classification features from each tissue is input into the combined analysis.

In certain embodiments, the methods comprise obtaining a gene expression profile at a time when a hibernating animal is protected from a disease or condition and a gene expression profile is obtained at a time when the hibernating animal is not protected from the disease or condition. In certain embodiments, the gene expression profile reflects discrete body temperature intervals during the torpor-arousal cycle and time intervals before, during or after the mammal's hibernating cycle. In certain embodiments, the methods comprise, matching gene expression profiles from a plurality of samples from a tissue from a hibernating mammal to generate a common gene expression signature.

In certain embodiments, the generating of the gene expression signature comprises performing a method selected from the group consisting of: RNA-seq, quantitative PCR, ATAC-seq, epigenomic analysis (e.g., chromatin immunoprecipitation, and bisulfite modification), proteomic analysis, DNA sequencing, and combinations thereof.

In certain embodiments, gene expression profiles between timepoints when hibernators are protected and not protected from diseases and conditions are compared to gene expression profiles from various human cell lines before and after exposure to various compounds using LINCS-L1000 data (Koleti, A. et al. “Data Portal for the Library of Integrated Network-based Cellular Signatures (LINCS) program: integrated access to diverse large-scale cellular perturbation response data” Nucleic Acids Res. (2018).

### Gene Network Analysis and Data Integration

In certain embodiments, the disclosed methods can include performing gene network analysis and multi-omics and/or cross-species data integration. In certain embodiments, weighted correlation network analysis, or weighted gene co-expression network analysis (WGCNA) (Langfelder, P. and Horvath, S. “WGCNA: an R package for weighted correlation network analysis” BMC Bioinformatics Vol. 9 2008) is performed. Such analysis generally produces a set of modules of size typically around 150 genes per module. In certain embodiments, another gene network analysis, gene regulatory network (GRN) inference analysis, is used to capture regulatory interactions between the genes among each module, using ARACNE (Margolin, et al., “ARACNE: An Algorithm for the Reconstruction of Gene Regulatory Networks in a Mammalian Cellular Context” BMC Bioinformatics 2006) and C3NET (Altay, G. et al., “Differential C3NET reveals disease netoworks of direct physical interactions” BMC Bioinformatics 20011) algorithms. Alternative methods include CLR (Faith, J. et al., “Large-scale mapping and validation of Escherichia coli transcriptional regulation from a compendium of expression profiles” PLOS Biology; 2007) and GENIE3 (Huynh-Thu, A. et al., “Inferring regulatory networks from expression data using tree-based methods” PLOS ONE; 2010), or Ac3net (Altay, G “Directed Conservative Causal Core Gene Networks” BioRxiv Cold Spring Harbor Laboratory 2018). The results of different methods can be directly integrated to obtain a single GRN per module which may assist in capturing potential targets. Because the typical co-expression network module out of WGCNA has approximately 150 genes, this results in 11175 (150×(150-1)/2) initial co-expressed gene pairs to be filtered. The GRN algorithms is expected to reduce the number of interactions to around 300 per module. In certain embodiments, such modules and interactions are further filtered to detect a small list of potential drug targets.

In certain embodiments, significant genes obtained from ATAC-seq data that are active among the different hibernation states are integrated. In certain embodiments, for a hibernation state of interest, overlap enrichment analysis is performed using Fisher's exact test to find significant enrichments of the genes over the filtered network modules. This can help to filter out some of the modules for the hibernation state of interest and give a smaller list of modules based on the enrichments of the transcription factors.

In certain embodiments, an assessment of target gene overlap with human genomics is performed. For example, an analysis of human genome-wide association study (GWAS) data to identify the disease or condition that is associated with a candidate gene target can be performed.

In certain embodiments, the statistically significant genes from animal and human curated external transcriptomic, proteomic studies, and/or human GWAS analysis for related phenotypes is then integrated over the modules in the scoring process. In certain embodiments, statistically significant genes from hibernator evolutionary rate analyses (accelerated and highly conserved within hibernators) are integrated and overlapping genes are favored in the scoring process. In certain embodiments, each gene in all the remaining module is scored regarding the number of interactions it has in one neighbor distance (favoring hub genes) and in three step neighboring distance (favoring a large subnetwork). In certain embodiments, a unit number is added to the scoring if the genes of the counted interactions (of each gene) are also available in multi-omics Random Forests, ATAC-seq, proteomics, GWAS and also the DGE analysis results of the transcriptomic data for the hibernation state of interest. This provides a ranked list of potential drug targets, which can be assessed for further wet-lab experimental validation.

In certain embodiments, the methods comprise integrating gene network analysis with molecular interaction databases. In certain embodiments, information obtained from gene network analysis will be integrated with a network related database. In certain embodiments, the network related database comprises human and/or model organism molecular interaction data from public repositories. In certain embodiments, the network related database comprises gene expression data (e.g., gene expression changes) from cells following pharmacologic or genetic perturbation. In certain embodiments, the network related database is ConsensusPathDB. In certain embodiments, the network related database is the Library of Integrated Network-based Cellular Signatures (LINCS). In certain embodiments, gene regulatory network (GRN) inference analysis is performed. In certain embodiments, the GRN inference analysis is ARACNE or C3NET. In certain embodiments, candidate gene targets obtained from integrated gene network analyses are scored according to the number of interactions identified, the type of interactions identified, the sum of dependency scores, the transcription factor status, and combinations thereof.

### L1000 Expression Optimizer (LEO) Network Analysis Approach for Identification of Candidate Therapeutics

In certain embodiments, the methods disclosed herein comprise computing the association between an input gene expression signature, defined as a set of changes in gene expression, and compounds in a previously determined dataset to nominate candidate therapeutics that can induce or reverse the signature. In certain embodiments, the previously determined dataset comprises a database of gene expression profiles that characterizes a plurality of compounds by the gene expression changes the compounds induce in cell lines. In certain embodiments, the methods comprise computing the similarity between the input gene expression signature and each signature in a database using similarity metrics such as, but not limited to, a weighted connectivity score or inner products. In certain embodiments, the methods comprise normalization of similarity scores by comparing them to reference similarity distributions. In certain embodiments, the methods comprise generating a single answer for each compound-cell line pair tested and/or a single answer for each compound, and the normalized scores are summarized within and across cell lines using a set of order statistics compared to a reference distribution.

### Assessment of Genetic Evolutionary Rate in Hibernators (Accelerated and Conserved)

In certain embodiments, the methods disclosed herein comprise identifying a candidate gene targets from evolutionary rate. In certain embodiments, hibernator conservation is assessed by comparison of genetic conservation differences between hibernators and non-hibernators. Ancestral sequence reconstruction is performed with prequel (Hubisz, M. et al., “PHAST and RPHAST: Phylogenetic Analysis With Space/Time Models” Brief Bioinform. 2011) for every mammalian exonic conserved element. Ancestral reconstruction can also be performed with MrBayes (Huelsenbeck J. and Ronquist, F. “MRBAYES: Bayesian inference of phylogenetic trees” Bioinformatics (2001) 17(8): 745-5), PARANA (Patro, R. et al. “Parsimonious reconstruction of network evolution” Algorithms Mol Biol. (2012); 7:25.) , RASP (Yu et al. “RASP (Reconstruct Ancestral State in Phylogenies): A tool for historical biogeography,” Molecular Phylogenetics and Evolution (2015) 87: 46-49. , VIP (Arias, S. et al. “Spatial analysis of vicariance: a method for using direct geographical information in historical biogeography” Cladistics (2011) 27(6): 617-628.), or FastML (Ashkenazy, H. et al. “FastML: a web server for probabilistic reconstruction of ancestral sequences” Nucleic Acids Res. 2012). Genetic distance is calculated from ancestral sequence to each mammal and genes that are significantly more conserved in hibernators than in non-hibernators are identified. In certain embodiments, hibernator conserved sequences are identified with Forward Genomics methods such as Generalized Least Square and the Branch Method (Prudent, X. et al. “Controlling for Phylogenetic Relatedness and Evolutionary Rates Improves the Discovery of Associations Between Species' Phenotypic and Genomic Differences” Molecular Biology and Evolution(2016) 33 (8): 2135-2150.) In certain embodiments, elements that are accelerated in hibernators are identified. UCSC's neutral phylogenetic model for mammals is used and genes with accelerated substitution rates are identified with the phyloP software (Miller, W. et al., “28-Way Vertebrate Alignment and Conservation Track in the UCSC Genome Browser”, Genome Res; 2007 Dec; 17(12): 1797-80).

### In Vitro Validation of Candidate Gene Targets and Candidate Therapeutic Compounds

In certain embodiments, the methods described herein comprise validating a candidate gene target by contacting cells of an in vitro model of a human disease or condition with a compound that modulates the gene target. In certain embodiments, the methods described herein comprise contacting mammalian cells having a candidate gene target with a candidate compound, and measuring the gene activity of the cell. In certain embodiments, the methods described herein comprise contacting cells having the gene target with a candidate therapeutic; and determining if a desired cellular response is generated after contacting the cells with the candidate therapeutic compound.

In certain embodiments, the methods described herein comprise identifying a plurality of target genes that functionally interact in a common cellular pathway; wherein a candidate therapeutic compound changes the expression of at least one of the target genes of the cellular pathway or changes the activity of a protein expressed by at least one of the target genes in the cellular pathway.

In certain embodiments, the desired cellular response is selected from the group consisting of: reduced cell death, increased cell survival, decreased cell damage, reduced oxidative stress and decreased inflammation. In certain embodiments, the candidate gene target is associated with disease or condition is selected from the group consisting of ischemia-reperfusion injury, neurovascular diseases, stroke, neurodegeneration, Alzheimer's disease, traumatic brain injury, thromboembolic disease, peripheral vascular disease, cardiovascular disease, obesity, and diabetes, muscle atrophy, sarcopenia, muscle wasting, bone atrophy, osteoporosis, osteopenia, inflammation, suspended animation, seasonal affective disorder, hyperthyroidism, hypothyroidism, diseases stemming from increased intestinal permeability, chronic fatigue syndrome, chronic obstructive pulmonary disease (COPD), fibrotic diseases, idiopathic pulmonary fibrosis (IPF), non-alcoholic steatohepatitis (NASH), non-alcoholic fatty liver disease (NAFLD), and ionizing radiation.

In certain embodiments, the modulator of the candidate gene target or the candidate therapeutic compound is a virus, a small molecule, a nucleic acid, or a protein (e.g., and antibody). In certain embodiments, the nucleic acid is a plasmid DNA. In certain embodiments, the nucleic acid is an RNA. In certain embodiments, the modulator of the candidate gene target is antisense RNA. In certain embodiments, the modulator of the candidate gene target is an RNA, including but not limited to, siRNA, shRNA, miRNA, lncRNA, piRNA, tasiRNA, or crRNA. In certain embodiments, the modulator of the candidate gene target comprises at least one agent for gene manipulation by CRISPR interference (CRISPRi) or CRISPR activation (CRISPRa) to knockdown or overexpress candidate gene targets, respectively. In certain embodiments, the modulator of the candidate gene target comprises a guide RNA designed to target the gene's promoter region or the transcriptional start site. In certain embodiments, the modulator of the candidate gene target achieves gene silencing with nuclease-deactivated Cas9 (dCas9) fused with a transcriptional repressor as well as a guide RNA delivered into cells by lentiviral infection. In certain embodiments, to achieve gene activation, a transcriptional activator is used to fuse with dCas9.

In certain embodiments, the candidate therapeutic compound causes a cellular response of increased cell survival, reduced oxidative stress, reduced reactive oxygen species, decreased cell damage, decreased inflammation or combinations thereof, after the cells have been subjected to a condition selected from the group consisting of: hypoxia, starvation, low glucose, oxidative stress, and reduced temperature. In certain embodiments, the increased survival cell survival is caused by reduced apoptosis.

### In Vivo Validation of Candidate Gene Targets and Candidate Therapeutic Compounds

In certain embodiments, the methods described herein comprise administering a candidate therapeutic compound to a mammalian subject having a candidate gene target, and measuring the gene activity is a tissue obtained from the subject. In certain embodiments, the candidate gene target is associated with disease or condition is selected from the group consisting of ischemia-reperfusion injury, neurovascular diseases, stroke, neurodegeneration, Alzheimer's disease, traumatic brain injury, thromboembolic disease, peripheral vascular disease, cardiovascular disease, obesity, and diabetes, muscle atrophy, sarcopenia, muscle wasting, bone atrophy, osteoporosis, osteopenia, inflammation, suspended animation, seasonal affective disorder, hyperthyroidism, hypothyroidism, diseases stemming from increased intestinal permeability, chronic fatigue syndrome, chronic obstructive pulmonary disease (COPD), fibrotic diseases, idiopathic pulmonary fibrosis (IPF), non-alcoholic steatohepatitis (NASH), non-alcoholic fatty liver disease (NAFLD), and ionizing radiation. In certain embodiments, the methods described herein comprise administering a candidate therapeutic compound to a mammalian subject having a candidate gene target; and determining if a desired cellular response is generated after administration of the candidate therapeutic compound. In certain embodiments, the desired cellular response is selected from the group consisting of: reduced cell death, increased cell survival, decreased cell damage, reduced oxidative stress, decreased inflammation, increased survival, increased cognitive function, decreased symptoms of cardiovascular disease, decreased symptoms associated with Alzheimer's disease, decreased symptoms related to diabetes, and weight loss, decreased muscle and bone loss due to inactivity, decreased injury due to states of metabolic suppression and suspended animation.

In certain embodiments, the desired cellular response is associated with a reduction of indicators of obesity including, but not limited to, increased oxygen consumption rate, increased lipid oxidation, decreased adipogenesis, decreased adipocyte viability, decreased intracellular triglyceride content, decreased intracellular triglyceride accumulation, decreased pancreatic lipase activity, decreased expression of adipogenic genes, increased brown adipose tissue function (e.g., recruitment and activation of nonshivering thermogenesis), increased expression of thermoregulatory marker genes (e.g., UCP1), increased cell viability or proliferation of brown and beige adipocytes, or increased or decreased secretion of satiety hormones (e.g., leptin, ghrelin, GLP-1, CCK, and PYY). In certain embodiments, the desired cellular response is associated with indicators of cold storage organ preservation including, but not limited to, increased cell viability when stored at low temperatures (e.g, 4° C.), decreased reactive oxygen species (ROS) production, decreased oxidative stress, maintenance of intracellular ATP levels, inhibition of ATP depletion, maintenance of intracellular calcium levels, inhibition of intracellular calcium accumulation, maintenance of mitochondrial membrane potential, or inhibition of loss of mitochondrial membrane potential.

In certain embodiments, the desired cellular response is associated with a reduction of indicators of diabetes including, but not limited to, increased insulin sensitivity, increased expression of insulin-stimulated genes (e.g., IRS1, IRS2, GLUT1), increased glucose uptake, increased glucose consumption, increased glucose-stimulated insulin secretion, decreased pancreatic islet amyloid formation, increased pancreatic B-cell viability, inhibition of pancreatic B-cell dedifferentiation, decreased oxidative stress, decreased inflammation, or inhibition of alpha-amylase or alpha-glucosidase expression or activity. In certain embodiments, the desired cellular response is associated with a reduction of indicators of cardiovascular disease including but not limited to, decreased collagen deposition, decreased fibroblast differentiation into myofibroblasts, increased or decreased expression of Smad Binding Elements (SBE), increased cardiac contractile force generation or contractility, increased or enhanced maintenance of calcium handling, decreased hypertrophy (cell size), or decreased expression of cardiac hypertrophic marker genes. In certain embodiments, the desired cellular response is associated with a reduction of indicators of stroke including, but not limited to, increased neuronal cell viability, decreased Caspase activation (e.g., Caspases 3-7), decreased mitochondrial damage, decreased neuronal cell damage, or increased cognitive function.

In certain embodiments, the desired cellular response is associated with a reduction of indicators of neurodegeneration (e.g., Alzheimer's disease) including, but not limited to, increased neuronal connectivity, increased electrical conduction in neurons, increased cognitive function, decreased tau phosphorylation, increased viability of neurons with Aβ-induced toxicity, decreased gamma secretase activity, or decreased amyloid precursor protein (APP) processing.

In certain embodiments, the desired cellular response is associated with a reduction of indicators of muscle atrophy (e.g., sarcopenia) including, but not limited to, decreased protein degradation, decreased activation of ubiquitin-proteasome-dependent proteolysis pathway, increased protein synthesis, increased myoblast proliferation, decreased myoblast cellular senescence, increased myotube (cellular) size, or increased myogenic differentiation.

In certain embodiments, the desired cellular response is associated with a reduction of indicators of bone atrophy (e.g., osteoporosis) including, but not limited to, increased osteoblast differentiation or decreased osteoclast differentiation.

In certain embodiments, the desired cellular response is associated with a reduction of indicators of inflammation and diseases stemming from increased intestinal permeability, including but not limited to, decreased paracellular permeability, or increased tight junction integrity (e.g., increased expression of cell junction proteins).

In certain embodiments, the desired cellular response is associated with indicators of targeted temperature management including, but not limited to, core body temperature (in vivo), cellular metabolism (e.g., energy utilization), arterial acid-base levels, electrolyte levels, or blood gas levels.

In certain embodiments, the desired cellular response is associated with a reduction of indicators of suspended animation (e.g., synthetic torpor) including, but not limited to, decreased oxygen consumption rate, suppressed mitochondrial respiration, or decreased glucose utilization.

In certain embodiments, the desired cellular response is associated with indicators of reduced ischemia-reperfusion injury including, but not limited to, reduced cell death, increased cell survival, decreased cell damage, reduced oxidative stress, or reduced infarct volume.

In certain embodiments, the desired cellular response is associated with indicators of hyperthyroidism or hypothyroidism including, but not limited to, T3 levels, T4 levels, Thyrotropin Releasing Hormone (TRH) levels, Thyroperoxidase inhibition, Sodium/Iodide Symporter mediated uptake, Thyroxine-binding globulin (TBG) binding, Thransthyretin (TTR) Binding, Deiodinase activity, Thyroid hormone glucuronidation, Thyroid hormone sulfation, Thyroid Hormone Transmembrane Transporter activity or expression, or Thyroid receptor binding or trans-activation.

In certain embodiments, the desired cellular response is associated with indicators of reduced Chronic Fatigue Syndrome including, but not limited to, increased oxygen consumption rate, increased mitochondrial respiration, ATP production, or reversal of hypometabolic state.

In certain embodiments, the desired cellular response is associated with a reduction of indicators of chronic obstructive pulmonary disease (COPD) or idiopathic pulmonary fibrosis (IPF) including, but not limited to, reversal of airway remodeling (Talaei, F. et al. J. of Experimental Biology 2011 214: 1276-1282), reduced globlet cell hyperplasia, reduced MUC5AC positive cells, reduced squamous cell metaplasia (SCM), or reduced mucociliary dysfunction.

In certain embodiments, the desired cellular response is associated with a reduction of indicators of non-alcoholic steatohepatitis (NASH) or non-alcoholic fatty liver disease (NAFLD) including, but not limited to, decreased inflammation, decreased intracellular lipid content or decreased collagen deposition. .

In certain embodiments, the desired cellular response is associated with indicators of reduced ionizing radiation including, but not limited to, decreased DNA damage, increased DNA repair, or decreased cytotoxicity.

In certain embodiments, the candidate therapeutic compound is administered to a mammalian subject in a sufficient amount for treatment of: neuronal injury following a neurovascular disease,. neuronal injury following a stroke, a cardiovascular disease, peripheral vascular disease, Alzheimer's disease, neurodegeneration, traumatic brain injury, thromboembolic disease, obesity, diabetes, muscle atrophy, sarcopenia, bone atrophy, osteoporosis, inflammation, suspended animation, seasonal affective disorder, hyperthyroidism, hypothyroidism, diseases stemming from increased intestinal permeability, chronic fatigue syndrome, chronic obstructive pulmonary disease (COPD), fibrotic disease, idiopathic pulmonary fibrosis (IPF), non-alcoholic steatohepatitis (NASH), non-alcoholic fatty liver disease (NAFLD), and ionizing radiation.

In certain embodiments, the methods described herein comprise administering a pharmaceutical composition comprising a candidate therapeutic compound identified by the methods described herein, to a mammalian subject having a candidate gene target, identified by the methods described herein.

### Compounds

In certain embodiments, this application discloses therapeutic compounds identified by the methods described herein for the treatment of a disease or condition described herein.

In certain embodiments, the therapeutic compound is a modulator of a candidate gene target identified by a method described herein. In certain embodiments, the therapeutic compound is one of a small molecule, a nucleic acid or a protein (e.g., an antibody). In certain embodiments, the nucleic acid is RNA. In certain embodiments, the RNA is antisense RNA. In certain embodiments the RNA is shRNA. In certain embodiments, the therapeutic compound changes the expression of at least one gene target identified by a method described herein.

For nucleic acid therapeutic compounds, the term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

### Pharmaceutical Compositions

The therapeutic compounds of the invention can be formulated in pharmaceutical compositions. These compositions can comprise, in addition to one or more of the therapeutic compounds, a pharmaceutically acceptable excipient, carrier, buffer, stabiliser or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal, intracardiac routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives can be included, as required.

Therapeutic compounds comprising nucleic acids can be formulated by any method known in the art including, but not limited to, formulation with polymeric nanoparticles (e.g., cationic polymers), a lipid nanoparticles, and/or other hydrophobic moieties (e.g., cholesterol).

A polypeptide, antibody, nucleic acid, small molecule or other pharmaceutically useful compound according to the present disclosure that is to be given to an individual, administration is preferably in a “therapeutically effective amount” that is sufficient to show benefit to the individual. A “prophylactically effective amount” can also be administered, when sufficient to show benefit to the individual. The actual amount administered, and rate and time-course of administration, will depend on the nature and severity of protein aggregation disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T.E. Creighton, Proteins: Structures and Molecular Properties (W.H. Freeman and Company, 1993); A.L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B(1992).

### Example 1: Disease Phenotypes Relate to Hibernation Time Points

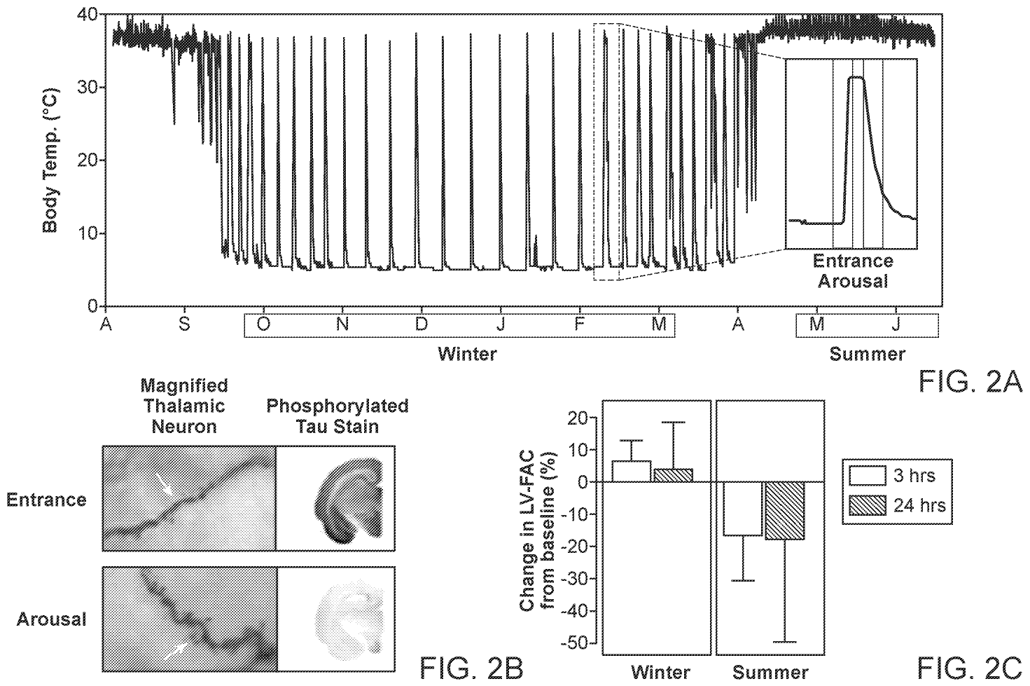

During hibernation of the model hibernator, the 13-lined ground squirrel, the body temperature was monitored to track phases of the hibernation cycle. Abdominal body temperature was recorded in 150 animals every 10 minutes from an implanted data logger to monitor the state of each animal during the year. Large temperature swings indicated cycles between torpor and arousal during hibernation (FIGS. 1 and 2A).

Neuronal protection from Alzheimer's Disease related-phenotypes in hibernators was observed in thalamic neurons (FIG. 2B). Tissues were stained for phosphorylated tau protein by performing immunohistochemistry using the AT8 monoclonal antibody. Neural microstructure was assessed as described in von der Ohe et al., Journal of Neuroscience, (2006) 26 (41) 10590-10598. During Entrance, neuronal spines retracted (white arrows) and hyperphosphorylated tau angles developed, and during Arousal, neuronal connections regrew and tau proteins were cleared. These results indicate that following hibernation, neurons of hibernators are protected from Alzheimer's Disease related phenotypes.

To assess ischemia-reperfusion phenotype protection in squirrels after hibernation, echocardiographic left ventricular systolic function (LV-FAC) was measured after surgically-induced ischemia reperfusion events in winter hibernating squirrels compared to summer non-hibernating squirrels (FIG. 2C). These results show that left ventricular systolic function is preserved in hibernating animals after ischemia reperfusion events compared to non-hibernating animals.

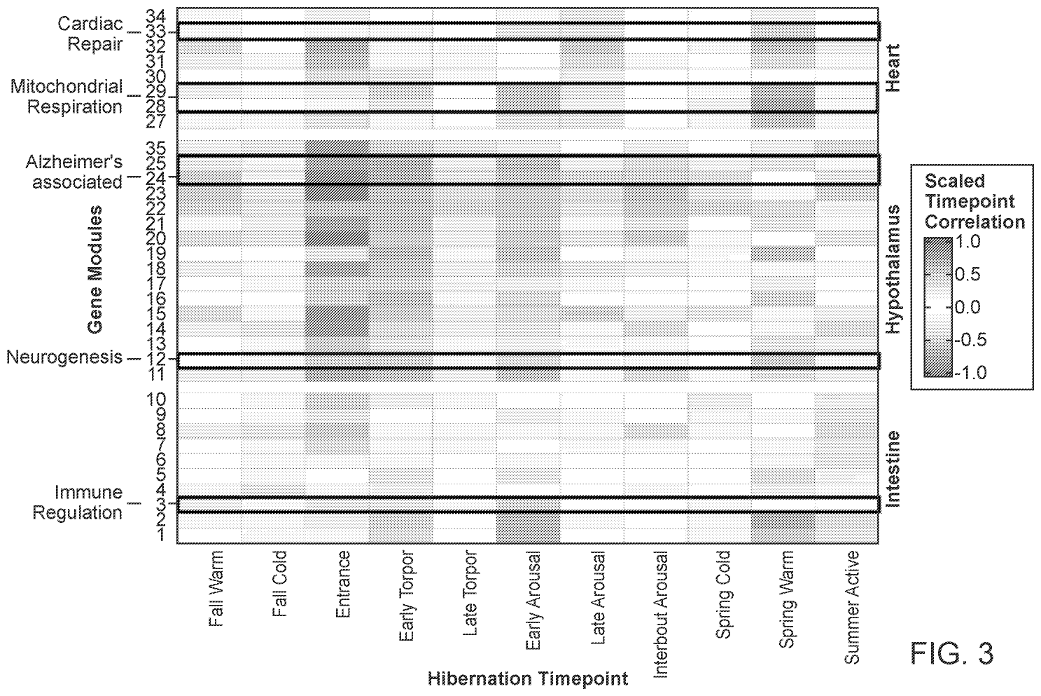

Gene expression profiles were generated from 3 tissues: heart, hypothalamus and intestines of 34 squirrels at different timepoints during the hibernation cycle using RNA-SEQ (FIG. 3). These results indicate that genes associated with various physiological mechanisms and pathologies vary greatly in expression throughout the hibernation cycle.

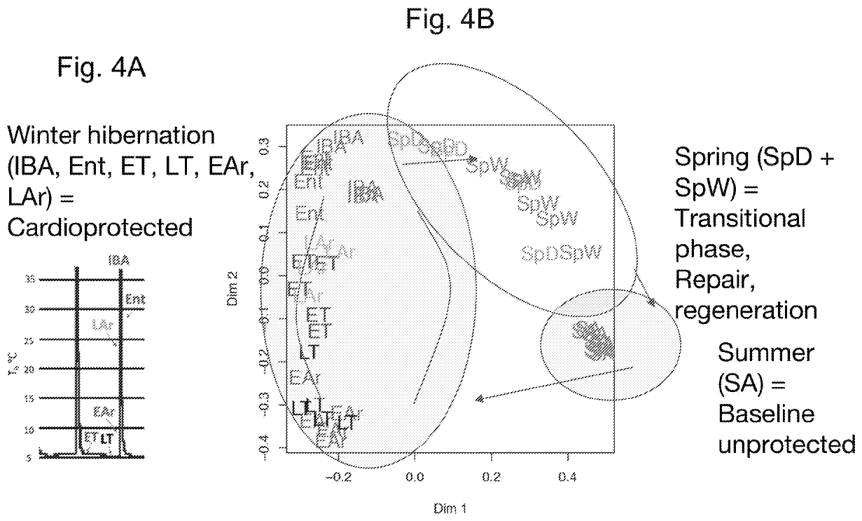

To verify if gene expression data obtained by RNA-SEQ from heart tissues of squirrels at different hibernation cycle timepoints was sufficient to observe differences in known cardiac protected gene signatures between timepoints, the gene signatures were obtained by RNA-SEQ from Squirrels in Winter (hibernating animals), Spring (transitional phase) and Summer (non-hibernating animals) (FIG. 4). RNASeq was performed on an Illumina HiSeq 2500 using ground squirrel heart sample libraries constructed from poly(A)-selected mRNA. For each library, approximately 20-30 million paired-end reads were generated, with each read consisting of 150 base-pairs. The reads were aligned to the 13-lined ground squirrel reference genome with HiSat2 and assembled into transcripts using StringTie. A merged reference transcriptome from all libraries was created with TACO, and gene expression abundances from each sample were estimated using StringTie and normalized using DESeq2.

These results show that transcriptomic data alone is sufficient to differentiate phenotypic groups that are cardioprotected and not cardioprotected.

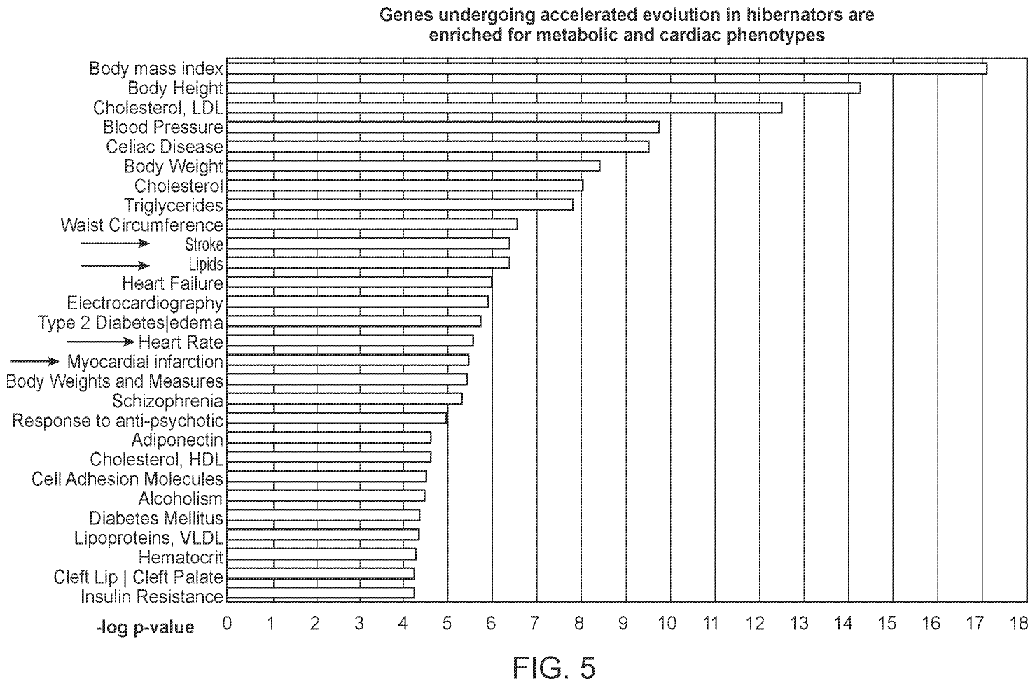

Genes in hibernating and non-hibernating animals were sequenced to identify the rate of evolution in particular genes associated with various physiological mechanisms or pathologies in hibernators compared to non-hibernating animals (FIG. 5). Accelerated evolution of groups of genes associated with cardiac and metabolic physiological mechanisms or pathologies was determined in hibernators compared to non-hibernating animals. UCSC's neutral phylogenetic model for mammals was used and genes with accelerated substitution rates were identified with the phyloP software (Pollard, K. et al., “Detection of Nonneutral Substitution Rates on Mammalian Phylogenies” Genome Res. 2010).

These results show that there is an evolutionary pressure in hibernating animals for adaptations in cardiac and metabolic mechanisms that can be used to identify novel gene targets that are important for conferring the protective phenotypes observed in hibernating mammals.

### Example 2: Target Identification Platforms

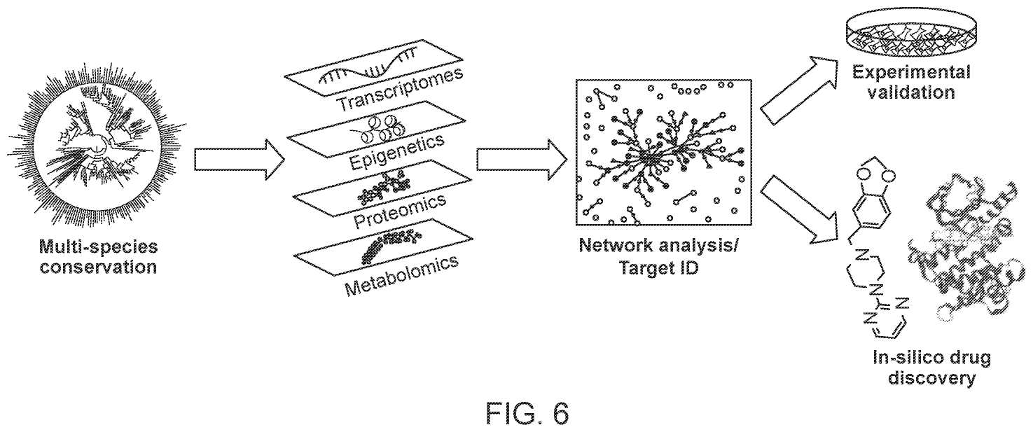

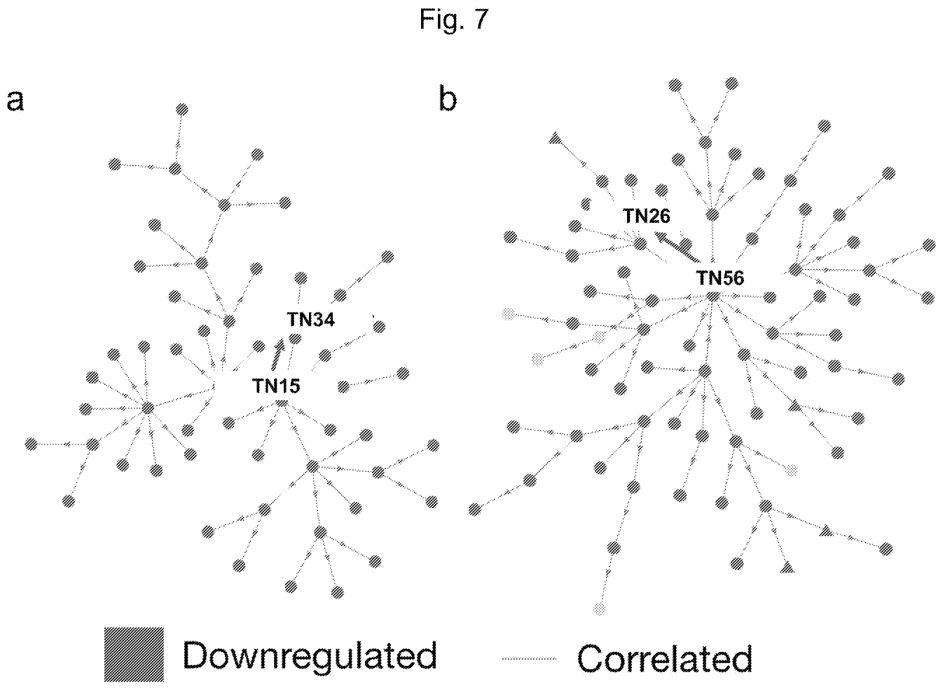

Novel target genes conferring protection from cardiac or neuronal ischemic reperfusion injury, or Alzheimer's disease were identified by integrating transcriptomic (RNA) expression analysis with at least one additional analysis comprising: proteomic (Protein); metabolomic, epigenetic and microbiome analysis to generate multi-omic integrated datasets of ground squirrel tissues obtained at timepoints during the hibernation cycle that confer protection and timepoints during the hibernation cycle that do not confer protection (FIG. 6). Following integration of datasets, computation models were generated comprising gene network analysis (Weighted Gene Compression Network Analysis) and Monarch with gene and phenotype association data across a plurality of species to identify candidate gene targets. Preliminary gene network modules were assembled for differentially expressed genes to predict correlated genes (FIG. 7). Candidate gene targets identified for cardioprotection are shown in Table 1. Candidate gene targets identified for neurological protection are shown in Table 2. FIG. 7 shows two sets of correlated candidate gene targets that were identified from the gene network modules. The candidate gene targets were then validated in vitro using established Alzheimer's Disease models or cardiac ischemia/reperfusion models (described below).

TABLE 1
Cardioprotection Gene Targets

| Target | | |
|---|---|---|
| Number | Gene | |
| (TN) | Symbol | Gene Name |
| 1 | ABAT | 4-Aminobutyrate Aminotransferase |
| 2 | ACSM5 | Acyl-CoA Synthetase Medium Chain Family |
| | | Member 5 |
| 3 | ADCY4 | Adenylate Cyclase 4 |
| 4 | ALDH6A1 | Aldehyde Dehydrogenase 6 Family Member A1 |
| 5 | AMD1 | Adenosylmethionine Decarboxylase 1 |
| 6 | ARAP3 | ArfGAP with RhoGAP Domain Ankyrin Repeat |
| | | and PH Domain 3 |
| 7 | BEX3 | Brain Expressed X-Linked 3 |
| 8 | BIRC3 | Baculoviral IAP Repeat Containing 3 |
| 9 | CEP85 | Centrosomal Protein 85 |
| 10 | CLK1 | CDC Like Kinase 1 |
| 11 | COQ9 | Coenzyme Q9 |
| 12 | CYP1A1 | Cytochrome P450 Family 1 Subfamily A Member |
| | | 1 |
| 13 | DBT | Dihydrolipoamide Branched Chain Transacylase |
| | | E2 |
| 14 | DVL2 | Dishevelled Segment Polarity Protein 2 |
| 15 | ECHDC2 | Enoyl-CoA Hydratase Domain Containing 2 |
| 16 | FH | Fumarate Hydratase |
| 18 | HADH | Hydroxyacyl-CoA Dehydrogenase |
| 19 | HSD17B10 | Hydroxysteroid 17-Beta Dehydrogenase 10 |
| 20 | ING3 | Inhibitor Of Growth Family Member 3 |
| 21 | INSIG2 | Insulin Induced Gene 2 |
| 22 | KCNJ2 | Potassium Inwardly Rectifying Channel |
| | | Subfamily J Member 2 |
| 23 | KLHDC2 | Kelch Domain Containing 2 |
| 24 | KMT2D | Lysine Methyltransferase 2D |
| 25 | LIG4 | DNA Ligase 4 |
| 26 | LMAN2L | Lectin, Mannose Binding 2 Like |
| 27 | LRIF1 | Ligand Dependent Nuclear Receptor Interacting |
| | | Factor 1 |
| 28 | LYSMD1 | LysM Domain Containing 1 |
| 29 | MAOB | Monoamine Oxidase B |
| 30 | MAX | MYC Associated Factor X |
| 31 | MCCC2 | Methylcrotonoyl-CoA Carboxylase 2 |
| 32 | MCL1 | MCL1 Apoptosis Regulator, BCL2 Family |
| | | Member |
| 33 | MEPCE | Methylphosphate Capping Enzyme |
| 34 | METTL7A | Methyltransferase Like 7A |
| 35 | MEX3C | Mex-3 RNA Binding Family Member C |
| 36 | MIS12 | MIS12 Kinetochore Complex Component |
| 37 | MON1A | MON1 Homolog A, Secretory Trafficking |
| | | Associated |
| 38 | PINK1 | PTEN Induced Kinase 1 |
| 39 | PLEKHA4 | Pleckstrin Homology Domain Containing A4 |
| 40 | PNRC2 | Proline Rich Nuclear Receptor Coactivator 2 |
| 41 | PTPN6 | Protein Tyrosine Phosphatase Non-Receptor Type |
| | | 6 |
| 42 | RAPGEF3 | Rap Guanine Nucleotide Exchange Factor 3 |
| 43 | RBM39 | RNA Binding Motif Protein 39 |
| 44 | RNF207 | Ring Finger Protein 207 |
| 45 | RNPC3 | RNA Binding Region (RNP1, RRM) Containing |
| | | 3 |
| 46 | SCRN3 | Secernin 3 |
| 47 | SMIM30 | Small Integral Membrane Protein 30 |
| 48 | SNRK | SNF Related Kinase |
| 49 | SRSF6 | Serine And Arginine Rich Splicing Factor 6 |
| 50 | SUOX | Sulfite Oxidase |
| 51 | TAB2 | TGF-Beta Activated Kinase 1 (MAP3K7) Binding |
| | | Protein 2 |
| 52 | TAF1D | TATA-Box Binding Protein Associated Factor, |
| | | RNA Polymerase I Subunit D |
| 53 | TNRC6B | Trinucleotide Repeat Containing Adaptor 6B |
| 54 | TOP1 | DNA Topoisomerase I |
| 55 | UBE2D4 | Ubiquitin Conjugating Enzyme E2 D4 (Putative) |
| 56 | UPF1 | UPF1 RNA Helicase And ATPase |
| 57 | XRCC5 | X-Ray Repair Cross Complementing 5 |
| 58 | ZC3H10 | Zinc Finger CCCH-Type Containing 10 |
| 59 | ZIK1 | Zinc Finger Protein Interacting With K Protein |
| | | 1 |
| 60 | ZKSCAN8 | Zinc Finger With KRAB And SCAN Domains 8 |
| 61 | ZNF646 | Zinc Finger Protein 646 |

TABLE 2
Neurological Protection Gene Targets

| Target | | |
|---|---|---|
| Number | Gene | |
| (TN) | Symbol | Gene Name |
| 62 | AK2 | Adenylate Kinase 2 |
| 63 | ASNSD1 | Asparagine Synthetase Domain Containing 1 |
| 64 | ATPAF2 | ATP Synthase Mitochondrial F1 Complex |
| | | Assembly Factor 2 |
| 65 | ATXN2 | Ataxin 2 |
| 66 | B4GALNT4 | Beta-1,4-N-Acetyl-Galactosaminyltransferase |
| | | 4 |
| 67 | BOD1 | Biorientation Of Chromosomes In Cell |
| | | Division 1 |
| 68 | BUD13 | BUD13 Homolog |
| 69 | CA2 | Carbonic Anhydrase 2 |
| 70 | CEACAM18 | CEA Cell Adhesion Molecule 18 |
| 71 | CIT | Citron Rho-Interacting Serine/Threonine |
| | | Kinase |
| 72 | CTDSP2 | CTD Small Phosphatase 2 |
| 73 | DNM1 | Dynamin 1 |
| 74 | DOK4 | Docking Protein 4 |
| 75 | DUSP6 | Dual Specificity Phosphatase 6 |
| 76 | EGLN2 | Egl-9 Family Hypoxia Inducible Factor 2 |
| 77 | FOXO3 | Forkhead Box O3 |
| 78 | FOXQ1 | Forkhead Box Q1 |
| 79 | HADC3 | Histone Deacetylase 3 |
| 80 | HDAC1 | Histone Deacetylase 1 |
| 81 | HDAC5 | Histone Deacetylase 5 |
| 82 | IFT20 | Intraflagellar Transport 20 |
| 83 | INHBA | Inhibin Subunit Beta A |
| 84 | INTS9 | Integrator Complex Subunit 9 |
| 85 | LBP | Lipopolysaccharide Binding Protein |
| 86 | MAX | MYC Associated Factor X |
| 87 | MED28 | Mediator Complex Subunit 28 |
| 88 | MESD | Mesoderm Development LRP Chaperone |
| 89 | MEX3C | Mex-3 RNA Binding Family Member C |
| 90 | MUC2 | Mucin 2, Oligomeric Mucus/Gel-Forming |
| 91 | PDK4 | Pyruvate Dehydrogenase Kinase 4 |
| 92 | POGK | Pogo Transposable Element Derived With |
| | | KRAB Domain |
| 93 | PPID | Peptidylprolyl Isomerase D |
| 94 | PPP1R12C | Protein Phosphatase 1 Regulatory Subunit 12C |
| 95 | RAB11FIP2 | RAB11 Family Interacting Protein 2 |
| 96 | RBM10 | RNA Binding Motif Protein 10 |
| 97 | RBM5 | RNA Binding Motif Protein 5 |
| 98 | RCC2 | Regulator Of Chromosome Condensation 2 |
| 99 | RNF24 | Ring Finger Protein 24 |
| 100 | RSBN1L | Round Spermatid Basic Protein 1 Like |
| 101 | SATB1 | SATB Homeobox 1 |
| 102 | SLC12A5 | Solute Carrier Family 12 Member 5 |
| 103 | SMAD5 | SMAD Family Member 5 |
| 104 | SNAPC3 | Small Nuclear RNA Activating Complex |
| | | Polypeptide 3 |
| 105 | SNCB | Synuclein Beta |
| 106 | TMEM2 | Cell Migration Inducing Hyaluronidase 2 |
| 107 | TNRC6B | Trinucleotide Repeat Containing Adaptor 6B |
| 108 | TRIM27 | Tripartite Motif Containing 27 |
| 109 | UPF1 | UPF1 RNA Helicase And ATPase |
| 110 | ZNF221 | Zinc Finger Protein 221 |
| 111 | ZNF646 | Zinc Finger Protein 646 |

### Example 3: Annotation Phenotypes for 11 Species Important for Alzheimer's Disease and Ischemia-Reperfusion Injury for Use in Target Validation

Data is curated from differential gene expression studies in the 13-lined ground squirrel (Ictidomys tridecemlineatus), that were conducted at precise hibernation time points (FIG. 1), as well as from an additional 10 species chosen due to their potential as models for either cardiac ischemia or Alzheimer's disease, or both, from publicly available datasets (Urocitellus parryii, Marmota monax, Heterocephalus glaber, Oryctolagus cuniculus, Cavia porcellus, Canis lupus familiaris, Sus scrofa, Macaca fascicularis, Macaca mulatta, Octodon degus). Associations to disease or phenotype traits and supporting evidence and provenance for these associations are captured by the curation process. Functional evidence for genes in these diseases, which still remain largely genetically uncharacterized are provided by phenotype annotations. The curated data is integrated into the resources of the Monarch Initiative, translating phenotype data available for non-model organisms and integrating them into structured phenotype descriptions already in Monarch using the following tools. RDF triples (Resource Description Framework data model to represent associations) are created using a Monarch Python package called ‘Dipper’. The transformed data is integrated into the Monarch graph database (Neo4J) using the SciGraph loader. A Representational State Transfer (REST) interface for performing semantic similarity matching of phenotypes is created to output a variety of similarity metrics given an input set of phenotypes.

### Example 4: Prioritization of Gene Candidates by Integrating Previously Identified Hibernator Disease-Resistance Gene Networks with Human and Newly-Annotated Animal Data

The accuracy of preliminarily identified Gene Networks were improved by integrating known molecular interactions and validating potential target genes across other animals. Integrating the gene network inference analysis with molecular interaction databases significantly improves the quality and accuracy of an inferred network. Interaction information was integrated from the two main network related databases: ConsensusPathDB that consists of a comprehensive collection of human (as well as mouse and yeast) molecular interaction data from 32 different public repositories, and a database comprising a library of network-based gene signatures that profiles gene expression changes following pharmacologic or genetic perturbation of cell lines in high-throughput (approximately 20,000 compounds, 4500 knockdowns, and 3000 over-expressions).

Using the interaction databases discussed above, gene pairs were retained for further analysis if they appear in the databases. For example, each of the disease-resistance gene network modules has approximately 150 genes, which would have 11175 (150× (150-1)/2) initial co-expressed gene pairs to be filtered. If very few gene pairs are removed after this filtering, the results were narrowed down by further filtering for only protein interactions in these databases. To keep potential novel regulatory interactions in the network modules, gene regulatory network (GRN) inference analysis using ARACNE and C3NET was performed over all module genes. The two combined GRNs with the filtered network described above were then integrated. After integration, each of the remaining genes were scored with respect to their number of interactions, type of interactions, sum of dependency scores, and transcription factor status.

Gene network modules were identified with the WGCNA package, which uses a topological overlap matrix to define the similarity in expression patterns between genes. Networks with regulation timing that matched the expectation for each disease-related phenotype.

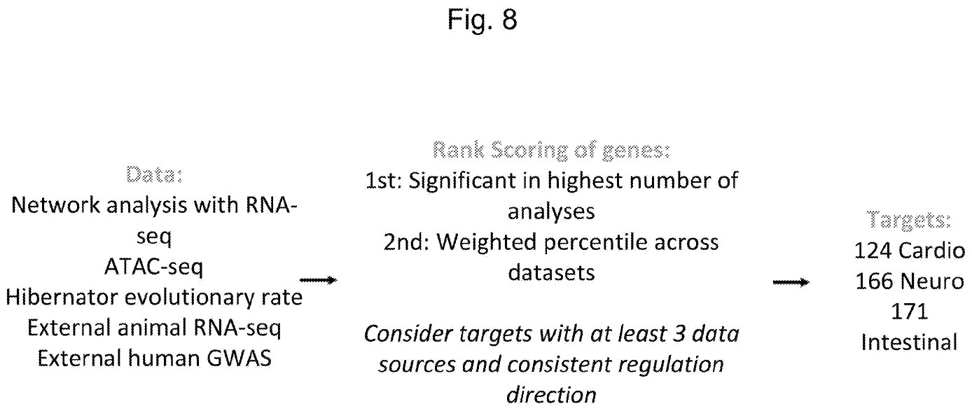

Evolutionary rate analysis was also performed. Statistically significant genes from hibernator evolutionary rate analyses (accelerated and highly conserved within hibernators) were also integrated and overlapping genes were favored in the scoring process. Potential gene targets were then ranked by the number of datasets they are identified as being significant and their weighted mean percentile score across the datasets (FIG. 8). Only targets that are seen in at least three datasets and have a consistent regulation direction across analyses (e.g., upregulated in RNA-seq and open chromatin in ATAC-seq) are considered for further validation. A ranked list of the hub genes (along with their interaction targets) for each network module was created, which is passed to cross-species phenotype validation with the Monarch browser, including the newly-annotated species from Example 3 above. Ultimately, genes were selected for in vitro testing based on proximity to a network hub, cross-species evidence, and druggability according to the Drug Gene Interaction Database (DGIdb).

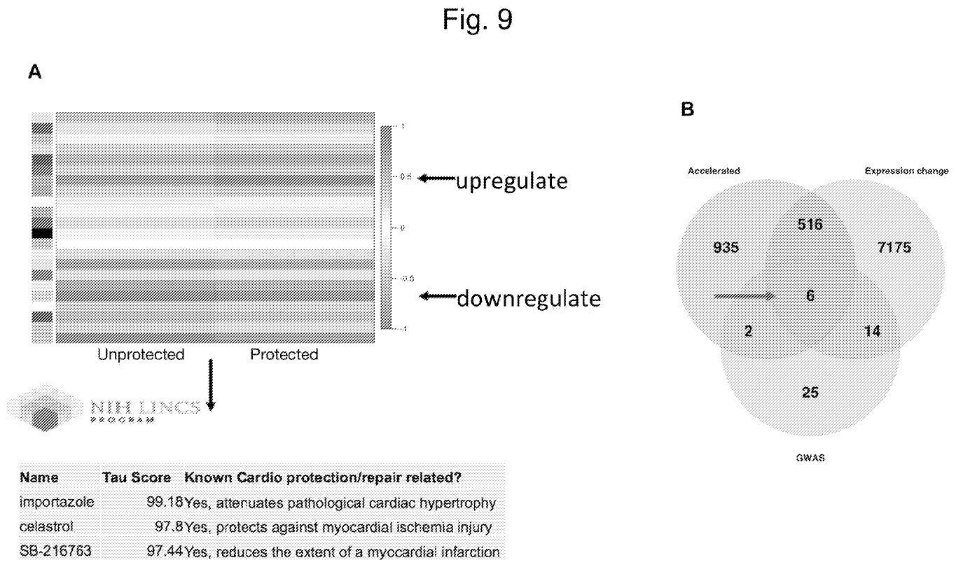

Using RNA-seq data from heart tissue of 60 animals across 3 timepoints (Arousal, Entrance, Summer), a gene network module with 156 genes was identified that is highly expressed specifically during the winter (Arousal, Entrance) while animals are protected from ischemia-reperfusion injury, but is downregulated during the Summer. Following integration as described above, the gene networks identified potential candidate gene targets to be validated in vitro (FIG. 9).

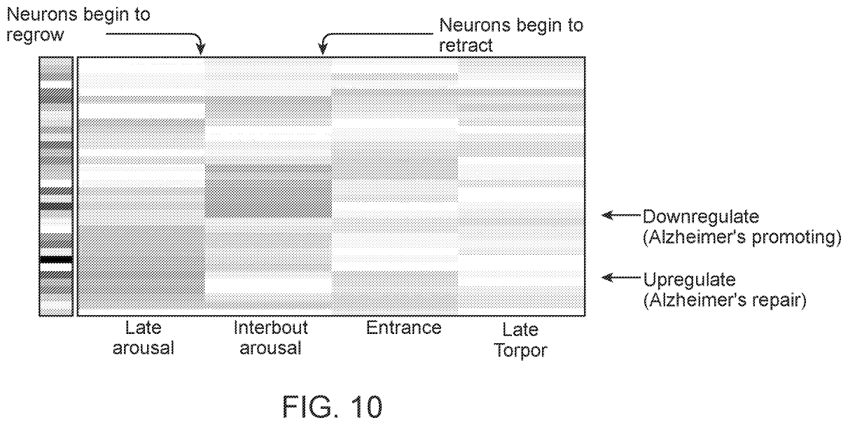

Using RNA-seq data from the brain tissue of 60 animals split across 3 timepoints (Arousal, Entrance, Summer), a gene network module of 143 genes was identified that is highly expressed as animals enter torpor while neuronal connections are lost and hyperphosphorylated tau tangles appear (FIG. 10). Another network module was inferred of 181 genes that is highly expressed during arousal as neuronal connections are re-growing and tau protein is cleared.

To verify the resulting networks, compounds from a database comprising a library of network-based gene signatures that profiles gene expression changes following pharmacologic or genetic perturbation of cell lines in high-throughput were identified that best matched the gene expression profiles of the hibernation networks discussed above (median connectivity tau score >95).

FIG. 9 shows select top scoring candidate therapeutic compounds obtained for cardiovascular protection from ischemia reperfusion injury, which included compounds known to be associated with protection from cardiac pathologies. Six out of 13 identified compounds have known beneficial effects on the heart, including celastrol, which is known to protect ischemic myocardium (FIG. 9).

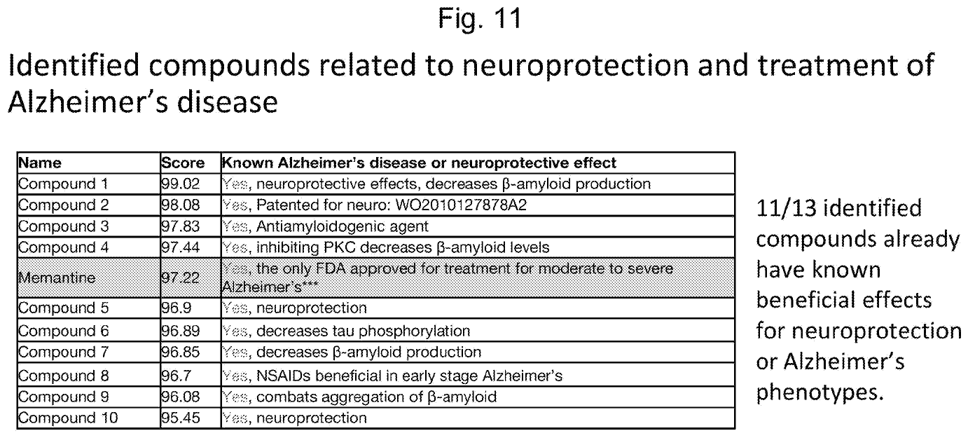

FIG. 11 shows select top scoring candidate therapeutic compounds obtained for protection from Alzheimer's disease, which included compounds known to be associated with neuroprotection. In searching for molecules that downregulate genes from the Alzheimer's induction mimicking network and upregulate genes from the Alzheimer's treatment mimicking network (FIG. 10), 11/13 identified compounds have known beneficial effects on Alzheimer's phenotypes, including memantine, the only drug to be FDA approved for moderate to severe Alzheimer's (FIG. 11).

These results show that the approach used for prioritization of target gene candidates by integrating previously identified hibernator disease-resistance gene networks with human and the newly-annotated animal data can successfully identify genes likely to be confer cardioprotection or neural protection.

### Example 5: In Vitro Validation of Top-Ranking Gene Targets for Ischemia-Reperfusion Injury

In vitro validation was performed for candidate gene targets preliminarily identified as described above. For in vitro validation of ischemia-reperfusion injury targets, an in vitro model of ischemia-reperfusion injury was used comprising stable cell lines over-expressing (OE) or under-expressing (KD) target genes using a lentiviral system. H9C2 rat cardiomyoblasts were purchased and grown in appropriate culture medium.

For knockdown experiments (KD), two independent SERPINH1 shRNA oligos were cloned into pLKO.1 plasmid vectors. Each shRNA plasmid was transfected together with a psPAX2 packaging plasmid and pMD2.G envelope plasmid into HEK-293T cells (ATCC) to create the lentiviral particles. These particles were purified and infected into cells. Cells stably expressing SERPINH1 shRNAs were selected using puromycin. Finally, the SERPINH1 knockdown was confirmed by qRT-PCR and western blot four days post-infection.

For gene overexpression (OE), cDNA clones (Origene) of the target genes are purchased and clone each into a pLenti-puro plasmid vector. Lentiviral particles were produced, H9C2 and SH-SY5Y cells were infected and selected. Gene overexpression was confirmed by qRT-PCR and western blot. Potential protection from injury was assessed by OE and wild-type (WT) Rat H9C2 myoblast cells, Human SH-SY5Y neuroblastoma cells, differentiated SH-SY5Y cells (mature neurons), and Human AC16 cardiomyocyte cells equally seeded into wells of control and experiment 96-well plates.

For ischemia-reperfusion assays, de-oxygenated serum free medium was prepared by adding 1% penicillin-streptomycin into glucose-free DMEM; the medium was contacted for 5 mins with 5% CO₂ and 95% N₂; and the medium was then warmed in a 37° C. bath. Cells were then washed with warm PBS to completely remove normal culture medium. PBS was aspirated and deoxygenated serum free medium was added into plates (100 μl was added for each well of 96-well plate). To mimic myocardial ischemia, cells in the experiment plate were subjected to ischemia by culturing them in glucose and serum free-medium and hypoxic conditions (5% CO₂ and 95% N₂). Plates were placed in a hypoxia chamber and air-tight lock; the chamber was filled with 5% CO₂ and 95% N₂ until oxygen level is 0%, and then the chamber was placed in the incubator for a duration of ischemia time. The duration of ischemia was 21 hours for H9C2 cells; 29 hours for differentiated SH-SY5Y cells; 16 hours for AC16 cells; and 16 hours for native SH-SY5Y cells. After ischemia, these cells were subjected to reperfusion by culturing them in normal conditions. The medium was replaced with normal culture medium for a duration of reperfusion. The reperfusion time was for 4 hours H9C2 cells; 18 hours for differentiated SH-SY5Y cells; 4 hours for AC16 cells; and 4 hours for native SH-SY5Y cells. Meanwhile, control cells were consistently cultured under normal conditions. Cell viability and cell damage was examined after ischemia/reperfusion injury for 17 hours with MTT and LDH assays. Cell viability was determined by MTT assay The control cells were considered to be 100% viable; changes in viability in the experiment plates were measured relative to the control.

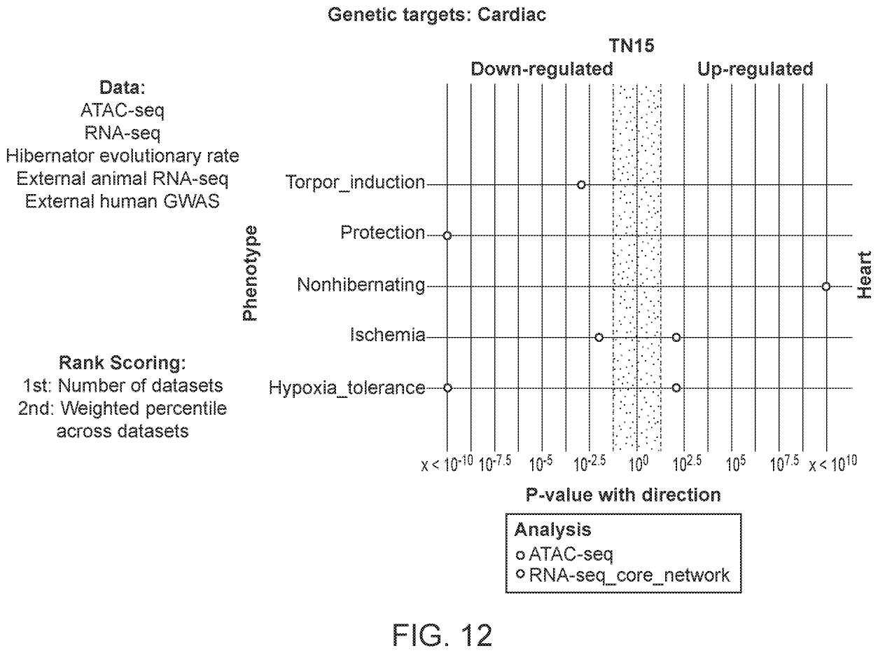

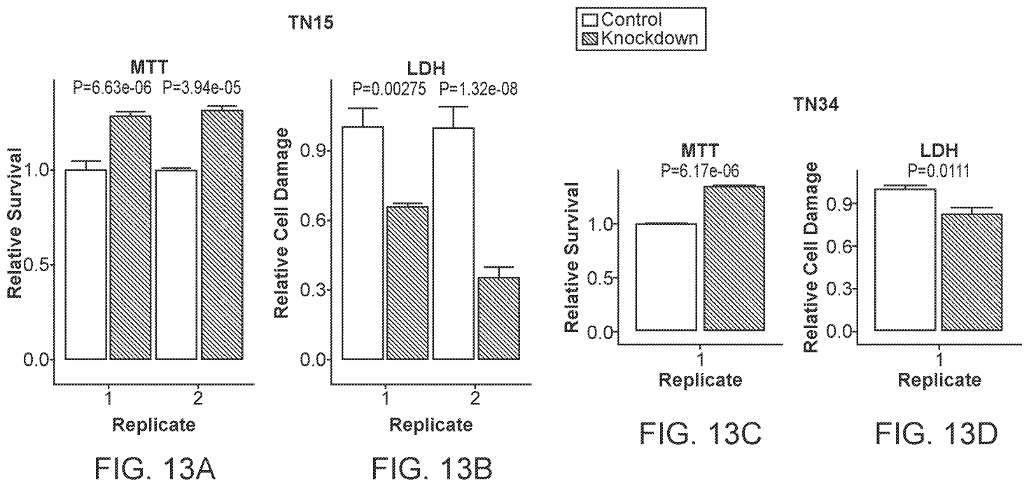

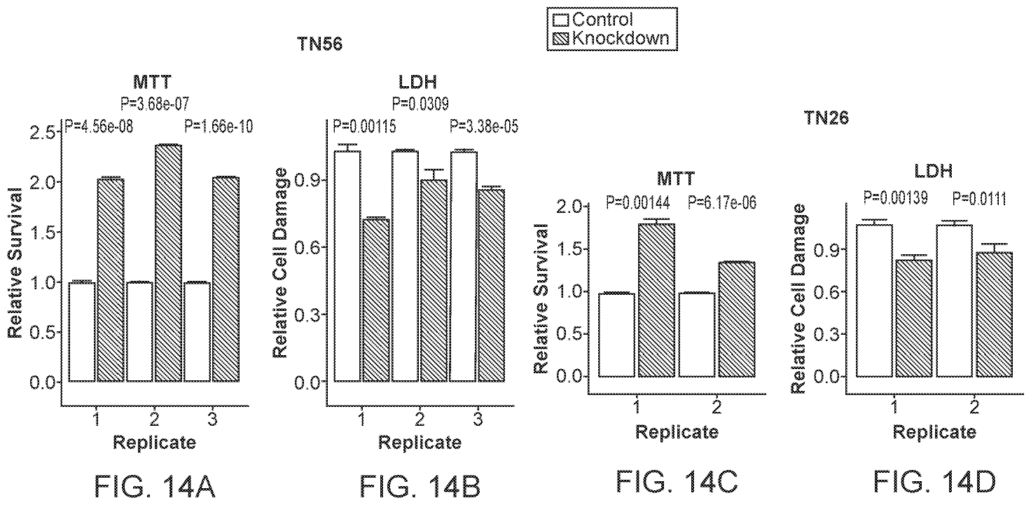

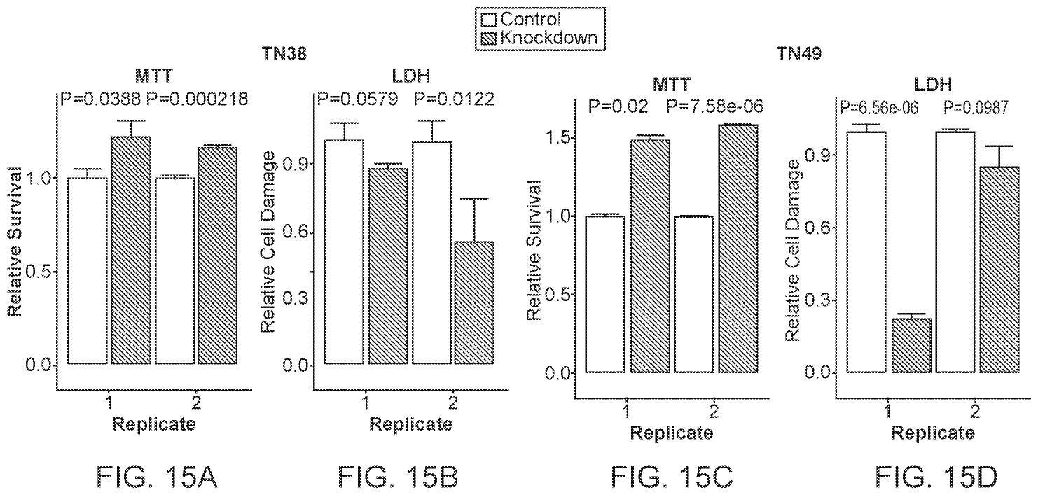

FIGS. 12-15 show the results of cell viability (MTT) assays and LDH activity assays performed in AC16 cells that have knockdown expression of select candidate gene targets (corresponding to the Target Number shown in Table 1) for cardioprotection following ischemia-reperfusion injury.

These results show that the candidate gene targets identified by the integrated gene networks obtained from the hibernator gene expression analysis and molecular interaction data sets described herein can be inhibited to confer protection of cardiac cells from ischemia-reperfusion injury in vitro.

### Example 6: In Vitro Validation of Top-Ranking Gene Targets for Alzheimer's Disease

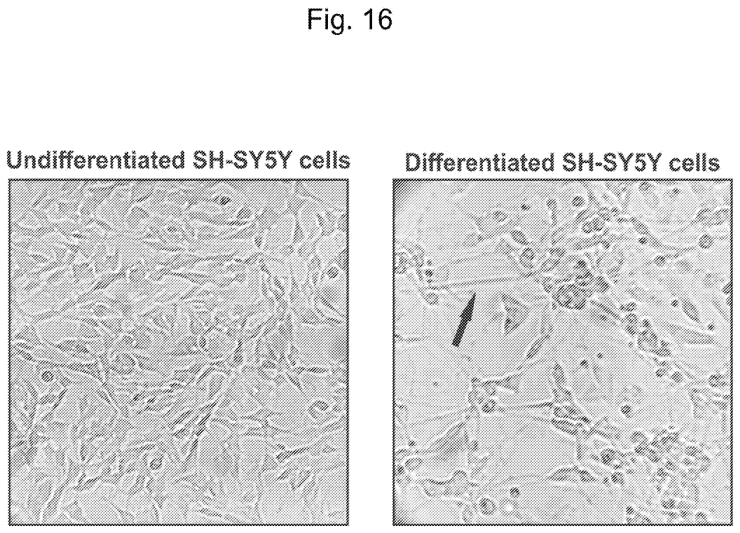

Alzheimer's disease progression features the accumulation of two protein aggregates: β-amyloid (Aβ) and tau; these play crucial roles in neuronal loss or dysfunction in Alzheimer's disease. To mimic pathogenesis of Alzheimer's disease, we used in vitro cell models that focus on Aβ-induced toxicity and tau hyperphosphorylation. SH-SY5Y human neuroblastoma cells (American Type Culture Collection; ATCC) were purchased and grown in appropriate culture medium. First, OE/KD and WT SH-SY5Y cells were equally seeded in wells of 96-well plates. Human SH-SY5Y neuroblastoma cells were successfully differentiated into mature neurons by incubating cells in retinoic acid and N₂ supplement for 9 days (FIG. 16). These can be distinguished from the original SH-SY5Y cells, which are prone to grow in clusters and extend short neurites at edges of the clusters. The differentiated SH-SY5Y cells do not cluster, have a more pyramidal shaped cell body, and long, extended neurites. Aβ-induced toxicity was examined by adding Aβ1-42 peptide to both WT and OE/KD cells. Aβ1-42 induced cell death in WT cells. The MTT assay was used to measure cell viability, and to determine whether the OE/KD cells showed enhanced protection against the cytotoxic effects of this peptide as compared to the WT cells.

Additionally, tau hyperphosphorylation is examined since it is the major event leading to tau aggregation. Tau hyperphosphorylation is induced by exposing the OE/KD and WT SH-SY5Y cells to hypothermic conditions (30° C.). Then, western blot is performed on cell lysates for tau phosphorylation sites to determine whether gene OE/KD reversed tau hyperphosphorylation as compared to WT in cells.

These results show that the integrated gene networks obtained from the hibernator gene expression analysis and molecular interaction data sets described herein can be used to identify gene targets that can be inhibited to confer protection of neuronal cells from injury in vitro.

### Example 7: Identification and Validation of Compounds That Confer Cardioprotection In Vitro

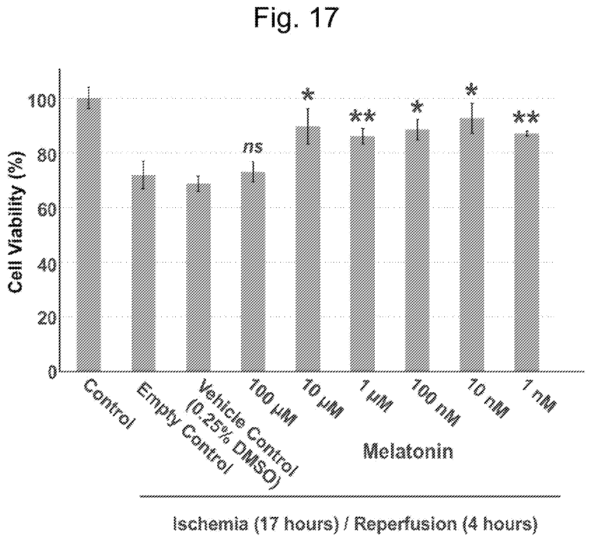

To validate potential cardioprotective compounds identified by the integrated gene networks described above, ischemia-reperfusion assays were conducted with H9C2 cells. Test compounds and vehicle controls (VC) were added into separate wells (DMSO, H₂O, EtOH). Control wells resulted in ˜75% viable cells (empty and vehicle controls), while melatonin, a positive control drug, protected cells from damage: here ˜90% cells were viable (FIG. 17)

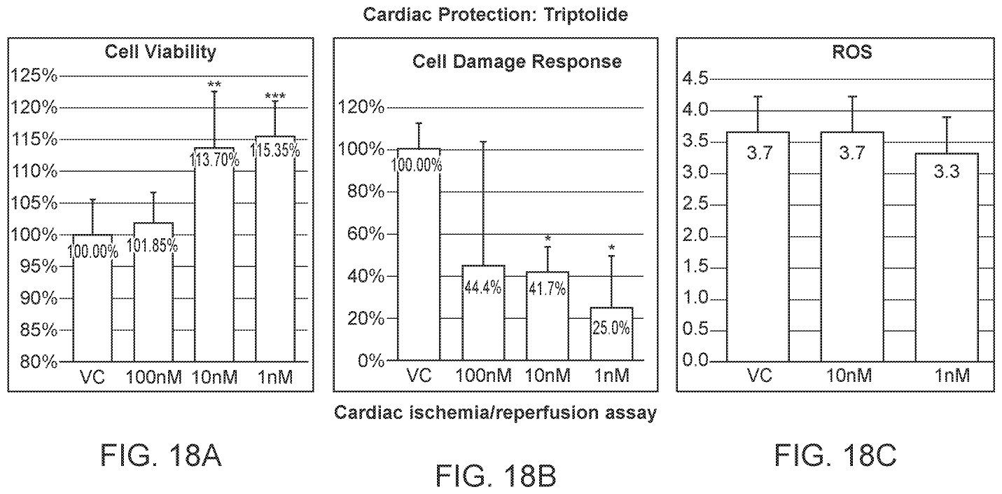

Ischemia-reperfusion assays were also performed in AC16 cells, and test compounds were added to the cells prior to ischemia (16 hours) and reperfusion (4 hours). A known cardioprotective compound, Triptolide, was identified which increased cell viability and reduced cell damage (FIG. 18).

These results confirm that the gene modules described herein can be used to identify and validate novel therapeutic compounds capable of conferring protection from cardiac ischemia-reperfusion injury in vitro.

### Example 8: Identification and Validation of Compounds That Confer Neurological Protection In Vitro

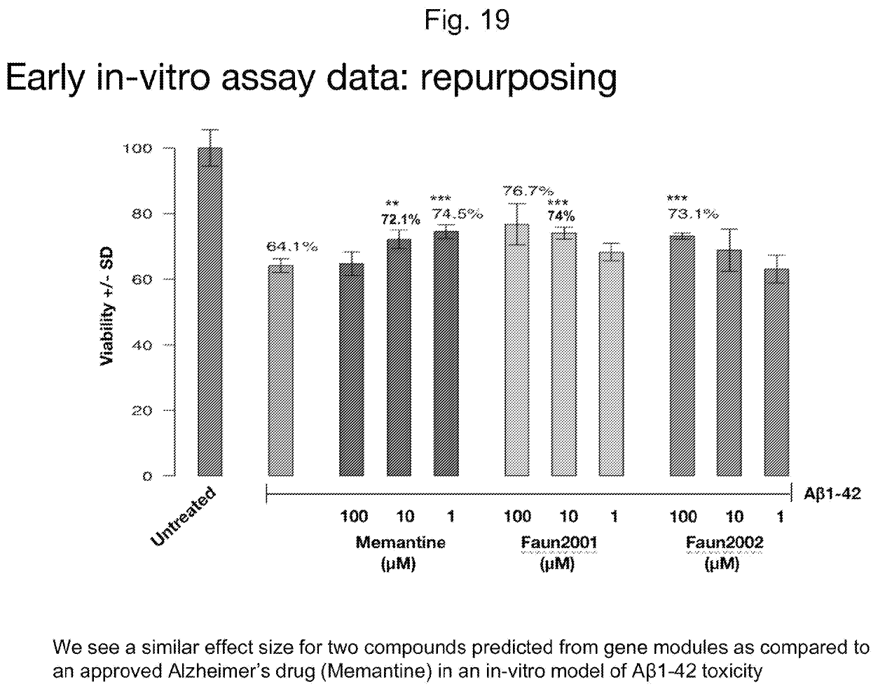

To validate potential neuroprotective compounds identified by the integrated gene networks described above, in vitro assays were conducted with SH-SY5Y cells. Aβ-induced toxicity was examined by adding Aβ1-42 peptide to cells treated with vehicle control or test compounds (FIG. 19). The MTT assay was used to measure cell viability, and to determine whether the cells incubated with the test compound showed enhanced protection against the cytotoxic effects of this peptide as compared to the vehicle control cells. Tau hyperphosphorylation was also examined, by exposing the SH-SY5Y cells to hypothermic conditions (30° C.). An increase in cell viability was observed for two of the test compounds identified from the gene modules, to a similar extent as Memantine, an approved Alzheimer's drug.

These results confirm that the gene modules described herein can be used to identify and validate novel therapeutic compounds capable of conferring protection from neuronal injury in a Alzheimer's Disease model in vitro.

### Example 9: Identification of Gene Targets and Therapeutic Compounds for Metabolic Conditions and Diseases, Sarcopenia and Bone Atrophy

The methods described herein are used for detection of novel gene targets and therapeutic compounds for the treatment of metabolic conditions and diseases, including, but not limited to, diabetes and obesity, and conditions and diseases associated with sarcopenia and bone atrophy.

The candidate gene targets and therapeutic compounds are validated in vitro using cell models of diabetes and obesity (e.g., lipogenesis), sarcopenia and bone atrophy and in mammalian subjects in vivo.

### Example 10: L1000 Expression Optimizer (LEO) Network Analysis Approach for Identification of Candidate Therapeutics

Using protective gene expression signatures discussed above for Gene Network Analysis and Data Integration, tool compounds were identified using a novel computational approach called LEO: L1000 Expression Optimizer. The approach utilizes the L1000 2020 data from the NIH's Library of Integrated Network-Based Cellular Signatures (LINCS).

The L1000 dataset is a large database of gene expression profiles that characterizes over 34,000 compounds by the gene expression changes they induce in cancer cell lines. The goal of LEO is to compute the association between an input gene expression signature, defined as a set of changes in gene expression, and the compounds in the L1000 dataset to nominate candidate therapeutics that can induce or reverse the signature. The underlying hypothesis of perturbational datasets like L1000 is that compounds which produce similar gene expression changes have a similar biological function.

LEO builds on the approach described in Subramanian, A. et al. (2017), and incorporates innovative analytical methods. First, the tool computes the similarity between the input gene expression signature and each signature in the L1000 database using similarity metrics like the weighted connectivity score and inner products. Next, the tool normalizes similarity scores by comparing them to reference similarity distributions. Finally, to give a single answer for each compound-cell line pair and a single answer for each compound, the normalized scores are summarized within and across cell lines using a set of order statistics compared to a reference distribution. LEO employs a novel family of similarity metrics that incorporate weighted gene sets or signatures from external experiments, including cosine distance. These metrics enable greater precision when identifying candidate compounds. Furthermore, LEO allows researchers to incorporate prior knowledge by specifying gene, compound, and cell line subspaces most relevant to the problem of interest.

LEO produces for each compound a single score that incorporates both sensitivity and specificity of association. A compound with a strong positive score produces changes similar to the input signature, and one with a strongly negative score produces changes opposite to the input signature. This tool and the underlying L1000 dataset provides a powerful mechanism to identify therapeutics that are biologically relevant to a particular phenotype. LEO has flexibility on the gene space, the set of signatures for comparison, the distance function used, and the method of aggregation.

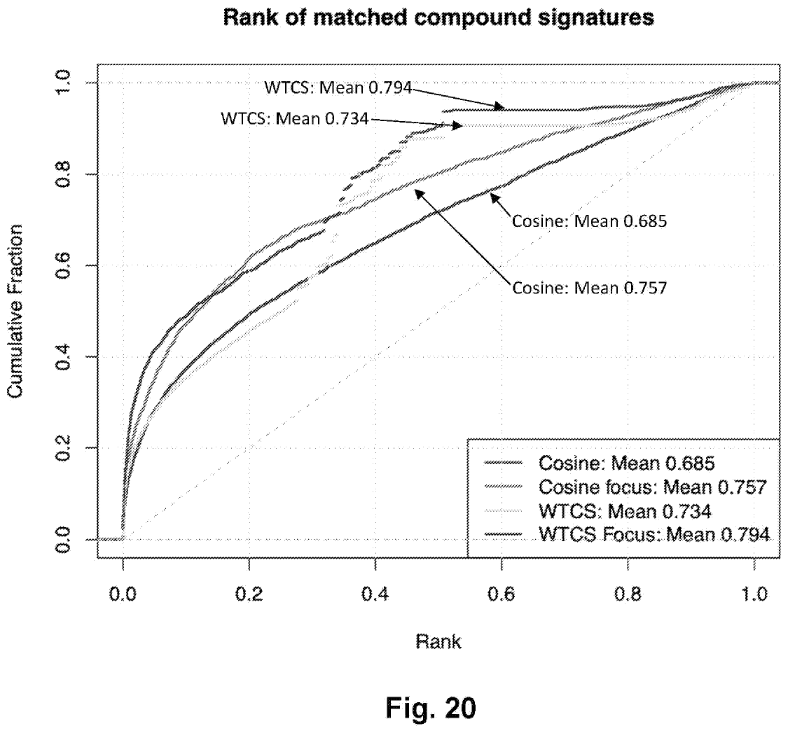

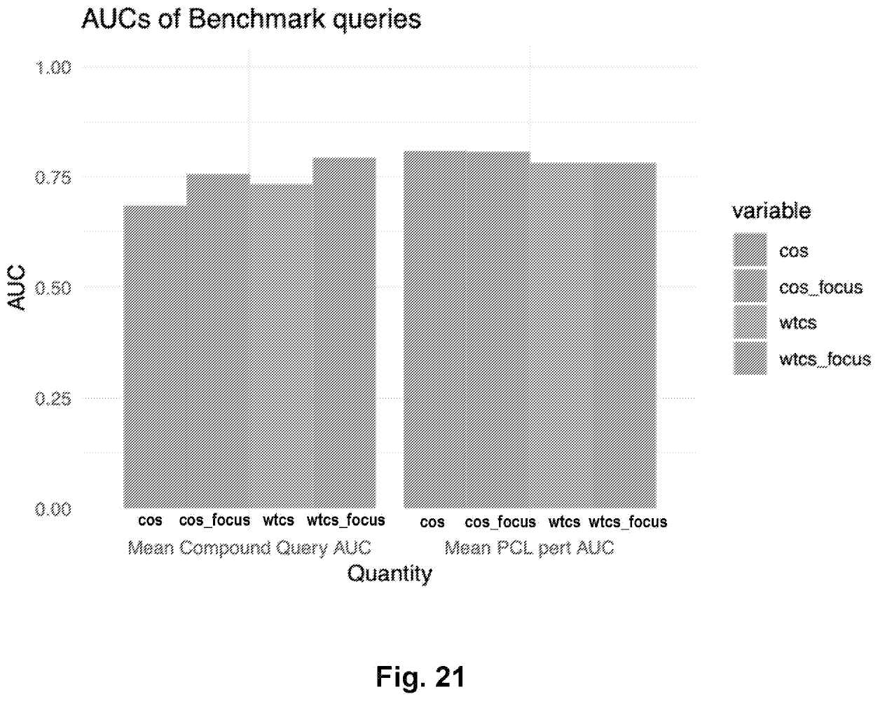

To evaluate the performance of LEO, a series of benchmarks were devised using L1000 signatures and known compound associations. For each of 25 randomly selected compound signatures in L1000, the entire L1000 database was queried using LEO and ranked signatures from most to least similar. Two of the metrics in LEO were evaluated-cosine distance and weighted connectivity score (WTCS). First, the ranks of those signatures of the same compound as the query signature irrespective of dose, time point, and cell line were assessed. Because of measurement noise, data quality, and the relevance of biological context, it was not expected that all signatures of a compound would agree. However, the queries were significantly enriched for signatures of matched compounds using four different LEO methods (FIG. 20). The areas under the ROC curves (auROC) for the LEO methods ranged from 0.685 to 0.794, with the most refined methods showing the best auROC (FIG. 21).

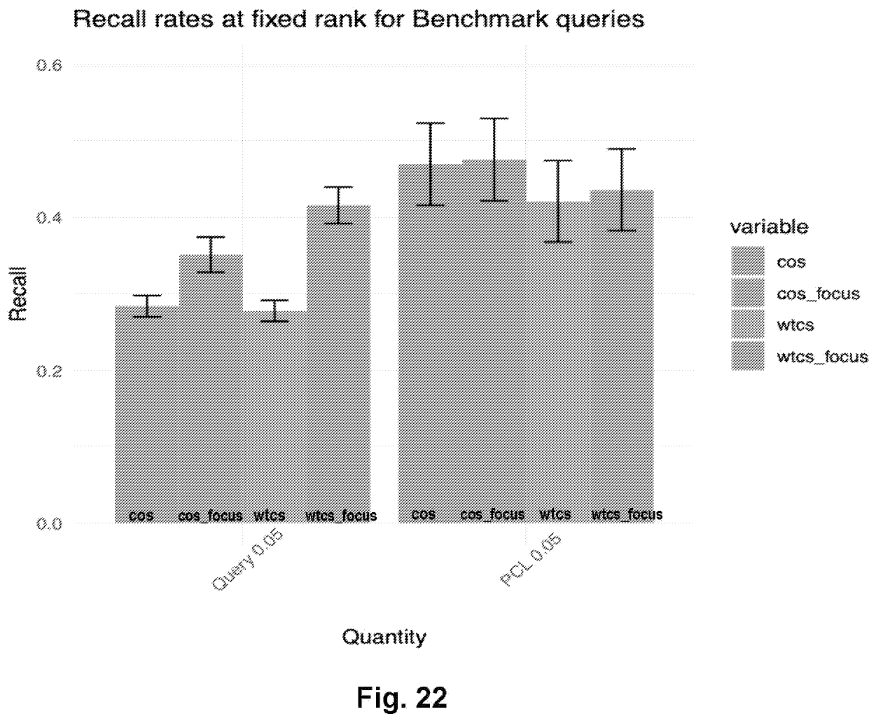

However, while matching the compound signature of interest is a robust benchmark with ground truth, it is not the most realistic use case because a typical input signature is derived from experiment rather than a known compound. Therefore, a more relevant benchmark was devised where the ranks of compounds belonging to the same pharmacological class (PCL) as the queried compound per external annotations published in Subramanian et al 2017 was identified. Using a compound signature to identify compounds with the same annotated mechanism of action can provide a more practical gauge of performance. To assess realistic use cases, recall at fixed sensitivity for two cases was quantified: (1) the fraction of same-compound signatures that were in the top 5% of the database and (2) the fraction of same-function compounds that were in the top 5% of query results summarized at the compound level. Within the top 5% of results, LEO methods recovered on average 33% of same-compound signatures, and 45% of PCL compounds (FIG. 22). Particularly given how important context can be, how noisy the data is, and that these benchmarks were based on one signature of a compound, this demonstrates that LEO can identify relevant compound signatures from the L1000 dataset.

### Example 11: Faun1003 Identified for Cardioprotection Using LEO Network Analysis and Validated for Cardioprotection In Vivo In Surgical I-R Model

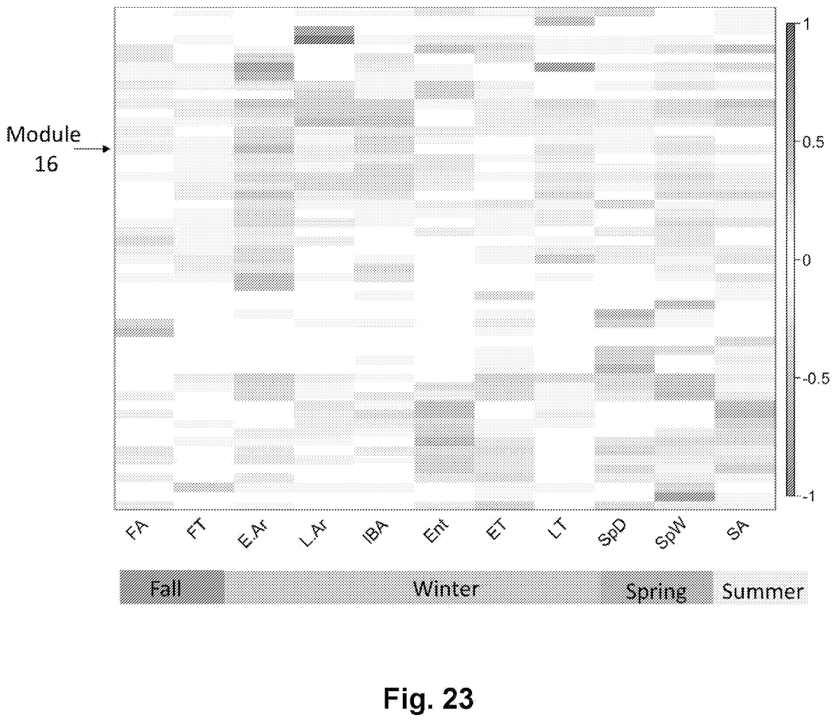

The upregulated genes from Module 16 were used as inputs into the analytical tool as described in Example 10 (FIG. 23). These module genes defined the gene expression signature and LEO was operated on level 5 or “signature” data using the “best-inferred genes” (BING) from the L1000 experiment. This method ranked the gene, Faun 1003, as fourth in the list for most closely mimicking the hibernator gene expression signature during cardioprotection.

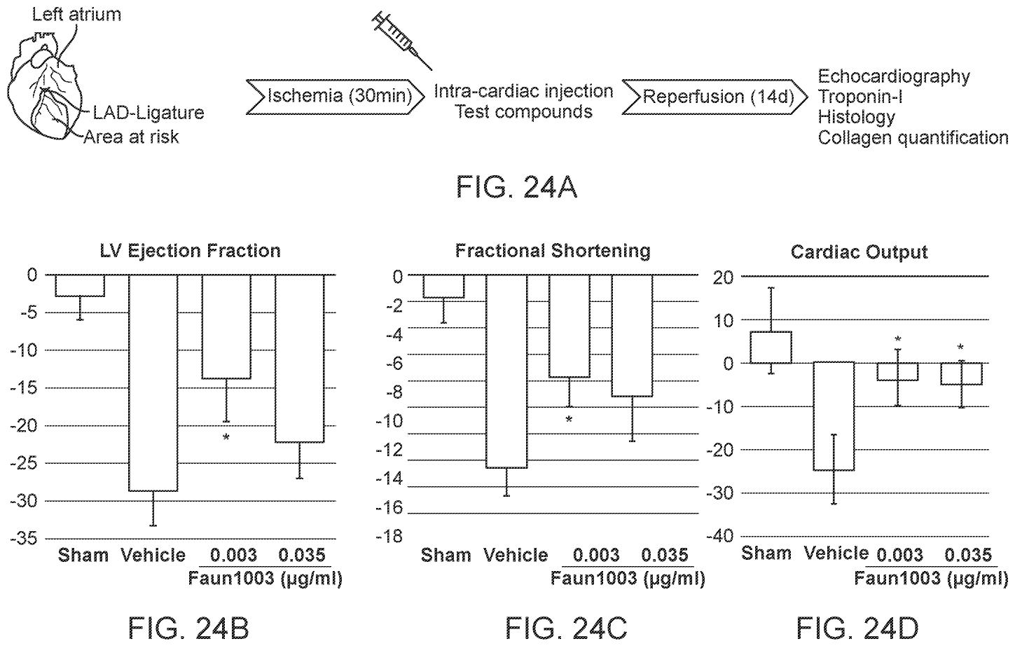

In addition to the in vitro work proposed in our Phase I project, this compound was tested in a surgical model of left coronary artery ligation. Animals (n=5/cohort) were subjected to 30 minutes of ischemia followed by intracardiac micro-injections (5 injections of 20 ul/injection administered at the border zone surrounding the area of blanching following LAD ligation) of 2 different doses of Faun1003, followed by removal of the coronary ligation allowing reperfusion. The volume and site of drug delivery by intracardiac injection was designed and optimized using Evans blue dye. In consecutive studies, various volumes of die were injected in areas surrounding the infarcted zone. Histologic examination of the tissues determined that volumes greater than 20 μl were detrimental to tissue organization, suggesting that larger volumes actually contributed to edema-like separation of the cardiac cells. In another series of studies, location of the injection was tested, also using histology to determine tissue spread, coverage within the infarcted zone, etc. These studies formed the basis for the injection protocol used in these experiments. Animals were observed for 14 days with echocardiography performed at 7 and 14 days post-surgery. Blood was obtained 24 hours post-surgery for quantification of Troponin I (FIG. 24a). Hearts were harvested at 14 days post-surgery and cardiac tissues were processed for histopathology as well as quantification of infarct size. The results of these experiments demonstrated a marked maintenance of cardiac function in treated animals as demonstrated by significant improvements in left ventricular ejection fraction and fractional shortening as well as a near return to baseline for cardiac output (FIG. 24 b-d).

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### REFERENCES CITED

Arendt, T. et al. Reversible paired helical filament-like phosphorylation of tau is an adaptive process associated with neuronal plasticity in hibernating animals. J. Neurosci. 23, 6972-6981 (2003).

Dave, K. R., Christian, S. L., Perez-Pinzon, M. A. & Drew, K. L. Neuroprotection: lessons from hibernators. Comp. Biochem. Physiol. B Biochem. Mol. Biol. 162, 1-9 (2012).

MacCannell, A. D. V., Jackson, E. C., Mathers, K. E. & Staples, J. F. An improved method for detecting torpor entrance and arousal in a mammalian hibernator using heart rate data. J. Exp. Biol. 221, (2018).

Quinones, Q. J. et al. Proteomic Profiling Reveals Adaptive Responses to Surgical Myocardial Ischemia-Reperfusion in Hibernating Arctic Ground Squirrels Compared to Rats. Anesthesiology 124, 1296-1310 (2016).

Martin, S. L. Mammalian hibernation: a naturally reversible model for insulin resistance in man? Diab. Vasc. Dis. Res. 5, 76-81 (2008).

Andres-Mateos, E. et al. Impaired Skeletal Muscle Regeneration in the Absence of Fibrosis during Hibernation in 13-Lined Ground Squirrels. PLOS One 7, e48884 (2012).

Utz, J. C., Nelson, S., O'Toole, B. J. & van Breukelen, F. Bone strength is maintained after 8 months of inactivity in hibernating golden-mantled ground squirrels, Spermophilus lateralis. J. Exp. Biol. 212, 2746-2752 (2009).

Subramanian, A. et al. (2017). A Next Generation Connectivity Map: L1000 Platform and the First 1,000,000 Profiles, Cell., 171(6), pp. 1437-1452.

1. A method of identifying a therapeutic target that can be modulated to treat a disease or condition, comprising:
obtaining tissue derived from one or more of an hibernator mammal species;
generating a gene expression profile from each tissue;
matching the gene expression profile from each tissue of hibernator animal to generate a common gene expression signature;
identifying a candidate gene target from the common gene expression signature; wherein the candidate gene target is found to be differentially expressed in tissues from a hibernating animal obtained when the animal is protected from a disease or condition compared to tissues obtained from a hibernating animal at a time when the hibernating animal is not protected from the disease or condition.

2. The method of claim 1, wherein the tissue is harvested at a time during the mammal's hibernating cycle when the hibernating animal is protected from the disease or condition.

3. The method of claim 1 or 2, wherein a plurality of tissue samples are each harvested at discrete body temperature intervals during the torpor-arousal cycle and time intervals before, during or after the mammal's hibernating cycle.

4. The method of any one of claims 1-3, further comprising performing an assay, wherein the assay comprises contacting cells with a modulator of the candidate gene target and determining if a desired cellular response is generated.

5. The method of claim 4, further comprising identifying a gene as a therapeutic target for the disease or condition if the desired cellular response is observed after contacting the cells with the inhibitor; wherein the desired cellular response indicates reduced cellular indicators of the disease.

6. A method of identifying a therapeutic compound for treating a disease or condition, comprising:
obtaining a gene expression profile from one or more of a hibernator mammal species, wherein the gene expression profile reflects discrete body temperature intervals during the torpor-arousal cycle and time intervals before, during or after the mammal's hibernating cycle;
identifying a candidate gene target from the gene expression profile;
contacting mammalian cells having the candidate gene target with a candidate compound, and measuring the gene activity of the cell.

7. A method for identifying a therapeutic compound for the treatment of a disease or condition, comprising:
obtaining tissue derived from each of a plurality of hibernating animals; wherein the tissue is harvested at a time point during the animal's hibernating cycle when the hibernating animal is protected from the disease or condition;
generating a gene expression profile from each tissue;
matching the gene expression profile from each tissue of the plurality of hibernating animals to generate a common gene expression signature;
identifying at least one gene target from the common gene expression signature; wherein the gene target is found to be differentially expressed in the tissues from a hibernating animal obtained when the animal is protected from a disease or condition compared to tissues obtained from a hibernating animal at a time-point when the hibernating animal is not protected from the disease or condition;
contacting cells having the gene target with a candidate therapeutic compound; and
determining if a desired cellular response is generated after contacting the cells with the candidate therapeutic compound.

8. The method of any one of claims 1-7 wherein the disease or condition is selected from the group consisting of ischemia-reperfusion injury, stroke, neurodegeneration, Alzheimer's disease, cardiovascular disease, obesity, diabetes, muscle atrophy, sarcopenia, bone atrophy, osteoporosis, inflammation, suspended animation, seasonal affective disorder, hyperthyroidism, hypothyroidism, diseases stemming from increased intestinal permeability, chronic fatigue syndrome, chronic obstructive pulmonary disease (COPD), idiopathic pulmonary fibrosis (IPF), non-alcoholic steatohepatitis (NASH), non-alcoholic fatty liver disease (NAFLD), and ionizing radiation.

9. The method of any one of claims 1-8, wherein the disease or condition is cardiac disease.

10. The method of any one of claims 1-8, wherein the disease or condition is neuronal ischemia/reperfusion injury.

11. The method of any one of claim 1-8, wherein the candidate gene target is selected from a gene in Table 1 or Table 2.

12. The method of any one of the above claims, wherein the generating the gene expression signature comprises performing RNA SEQ.

13. The method of claim 4, wherein the generating the gene expression signature further comprises performing a method selected from the group consisting of: epigenomic analysis, ATAC-seq, proteomic analysis, quantitative PCR and DNA sequencing and combinations thereof.

14. The method of any one of the above claims, wherein the candidate gene target is undergoing accelerated evolution in at least one hibernating mammal as compared to non-hibernating mammals.

15. The method of any one of the above claims, wherein the candidate gene target is conserved in hibernating mammals as compared to non-hibernating mammals.

16. The method of any one of the above claims, further comprising analysis of human genome-wide association study (GWAS) data to identify the disease or condition that is associated with a candidate gene target.

17. The method of any one of the above claims, wherein the hibernating mammal is selected from the group consisting of: (Ictidomys tridecemlineatus, Urocitellus parryii, Marmota monax, Spermophilus dauricus, Tenrec ecaudatus, Cheirogaleus medius, Cheirogaleus crossleyi, Cheirogaleus sibreei, Dromiciops gliroides, Cercartetus nanus, Burramys parvus, Tachyglossus aculeatus, Mirza coquereli, Glis glis, Graphiurus murinus, Muscardinus avellanarius, Miniopterus schreibersii, Rhinolophus ferrumequinum, Zapus hudsonius, Mesocricetus auratus, Cricetus cricetus, Erinaceus europaeus, Ursus arctos, Ursus americanus, Miniopterus natalensis, Myotis brandtii, Eptesicus fuscus, Myotis myotis, Myotis brandtii, Myotis ricketti and Myotis lucifugus).

18. The method of any one of the above claims; wherein the obtaining of tissues from each of a plurality of hibernating mammals is from different species.

19. The method of any one of the above claims, wherein the desired cellular response is selected from the group consisting of: reduced cell death, increased cell survival, decreased cell damage, reduced oxidative stress and decreased inflammation.

20. The method of any one of the above claims, wherein the cells are human cells.

21. The method of claim 4, wherein the modulator of the candidate gene target is one of a small molecule, a nucleic acid or a protein.

22. The method of claim 21, wherein the nucleic acid is RNA.

23. The method of claim 22, wherein the RNA is antisense RNA.

24. The method of claim 6 or 7, wherein the candidate therapeutic compound changes the expression of at least one gene target.

25. The method of claim 24, wherein the gene target is selected from a gene in Table 1 or Table 2.

26. The method of claim 6 or 7, wherein the candidate therapeutic compound causes a cellular response of increased cell survival, reduced oxidative stress, reduced reactive oxygen species, decreased cell damage, decreased inflammation or combinations thereof, after the cells have been subjected to a condition selected from the group consisting of: hypoxia, starvation, low glucose, oxidative stress, and reduced temperature.

27. The method of claim 26, wherein the increased survival cell survival is caused by reduced apoptosis.

28. The method of claim 6 or 7, further comprising identifying a plurality of target genes that functionally interact in a common cellular pathway; wherein the candidate therapeutic compound changes the expression of at least one of the target genes of the cellular pathway or changes the activity of a protein expressed by at least one of the target genes in the cellular pathway.

29. The method of any one of claims 6-28, wherein the therapeutic compound is administered to a patient in a sufficient amount for treatment of neuronal injury following a stroke.

30. The method of any one of claims 6-28, wherein the therapeutic compound is administered to a patient in a sufficient amount for treatment of a cardiovascular disease.

31. The method of any one of claims 6-28, wherein the therapeutic compound is administered to a patient in a sufficient amount for treatment of Alzheimer's disease.

32. The method of claim 26, wherein the candidate therapeutic compound causes increased cell viability, reduced reactive oxygen species, reduced cell damage, reduced inflammation or combinations thereof in neurons, after the neurons have been subjected to hypoxic conditions.

33. The method of claim 32, wherein the increased cell viability is caused by decreased apoptosis.

34. The method of claim 6 or 7, wherein the candidate therapeutic compound causes increased cell survival in neurons subjected to Aβ1-42 toxicity.

35. A method of identifying a therapeutic target that can be modulated to treat a disease or condition, comprising:
a. computing an association between an input gene expression signature and compounds in a previously determined dataset to nominate candidate therapeutics that can induce or reverse the signature, wherein the input gene expression signature represents changes in gene expression in a cell line;
b. determining the similarity between the input gene expression signature and each signature in a database using similarity metrics;
c. normalizing the similarity scores by comparing them to reference similarity distributions; and
d. identifying a candidate gene target based on the normalized similarity score.

36. The method of claim 35, wherein the previously determined dataset comprises a database of gene expression profiles that characterizes a plurality of compounds by the gene expression changes the compounds induce in cell lines.

37. The method of claim 35 or 36, wherein the similarity metric comprises a weighted connectivity score or cosine distance.

38. The method of any one of claims 35-37, wherein the generating comprises generating a single answer for each compound-cell line pair tested.

39. The method of any one of claims 35-38, further comprising summarizing normalized scores within and across cell lines using a set of order statistics compared to a reference distribution.
